(19) United States
(12) Patent Application Publication
(10) Pub. No.: US 2007/0083186 A1
(43) Pub. Date: Apr. 12, 2007

(54) Transdermal drug delivery systems, devices, and methods employing novel pharmaceutical vehicles

(76) Inventors: Darrick Carter, Seattle, WA (US); Dale Kalamasz, Redmond, WA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/541,383

(22) Filed: Sep. 27, 2006

Related U.S. Application Data
(60) Provisional application No. 60/722,136, filed on Sep. 30, 2005.
(60) Provisional application No. 60/754,688, filed on Dec. 29, 2005.
(60) Provisional application No. 60/755,199, filed on Dec. 30, 2005.
(60) Provisional application No. 60/755,401, filed on Dec. 30, 2005.

(51) Int. Cl. A61M 31/00

(52) U.S. Cl. 604/501; 604/20; 424/449

(57) ABSTRACT

Systems, devices, and methods for transdermal delivery of one or more therapeutic active agents to a biological interface. An iontophoretic drug delivery system is provided for transdermal delivery of one or more therapeutic active agents to a biological interface of a subject. The iontophoretic drug delivery system includes at least one active agent reservoir. The one or more active agent reservoirs are loadable with a vehicle for transporting, delivering, encapsulating, and/or carrying the one or more active agents

### CROSS-REFERENCE TO RELATED APPLICATION

This application claims the benefit under 35 U.S.C. § 119(e) of U.S. Provisional Patent Application No. 60/722,136 filed Sep. 30, 2005; U.S. Provisional Patent Application No. 60/754,688 filed Dec. 29, 2005; U.S. Provisional Patent Application No. 60/755,199 filed Dec. 30, 2005; and U.S. Provisional Patent Application No. 60/755,401 filed Dec. 30, 2005; where these four provisional applications are incorporated herein by reference in their entireties.

### BACKGROUND

1. Field

This disclosure generally relates to the field of iontophoresis and, more particularly, to transdermal drug delivery systems, devices, and methods for delivering pharmaceutically acceptable vehicles including one or more active agents to a biological interface.

2. Description of the Related Art

lontophoresis employs an electromotive force and/or current to transfer an active agent (e.g., a charged substance, an ionized compound, an ionic a drug, a therapeutic, a bioactive-agent, and the like), to a biological interface (e.g., skin, mucus membrane, and the like), by applying an electrical potential to an electrode proximate an iontophoretic chamber containing a similarly charged active agent and/or its vehicle.

lontophoresis devices typically include an active electrode assembly and a counter electrode assembly, each coupled to opposite poles or terminals of a power source, for example a chemical battery or an external power source. Each electrode assembly typically includes a respective electrode element to apply an electromotive force and/or current. Such electrode elements often comprise a sacrificial element or compound, for example silver or silver chloride. The active 5 agent may be either cationic or anionic, and the power source may be configured to apply the appropriate voltage polarity based on the polarity of the active agent. lontophoresis may be advantageously used to enhance or control the delivery rate of the active agent. The active agent may be stored in a reservoir such as a cavity. See e.g., U.S. Pat. No. 5,395,310. Alternatively, the active agent may be stored in a reservoir such as a porous structure or a gel. An ion exchange membrane may be positioned to serve as a polarity selective barrier between the active agent reservoir and the biological interface. The membrane, typically only permeable with respect to one particular type of ion (e.g., a charged active agent), prevents the back flux of the oppositely charged ions from the skin or mucous membrane.

Commercial acceptance of iontophoresis devices is dependent on a variety of factors, such as cost to manufacture, shelf life, stability during storage, efficiency and/or timeliness of active agent delivery, biological capability, and/or disposal issues. Commercial acceptance of iontophoresis devices is also dependent on their ability to deliver drugs across various biological interfaces including, for example, tissue barriers. For example, it may be desirable to have novel approaches for overcoming the poor permeability of skin.

The present disclosure is directed to overcome one or more of the shortcomings set forth above, and provide further related advantages.

### BRIEF SUMMARY

In one aspect, the present disclosure is directed to an iontophoretic drug delivery device for providing transdermal delivery of one or more therapeutic active agents to a biological interface. The device includes an active electrode assembly including at least one active electrode element, and at least one active agent reservoir.

The at least one active agent reservoir includes a pharmaceutically acceptable vehicle for transporting one or more active agents. In some embodiments, the pharmaceutically acceptable vehicle includes at least one surfactant, at least one nonpolar solvent, and at least one polar agent.

In some embodiments, the at least one active electrode element is operable to provide an electromotive force for driving the pharmaceutically acceptable vehicle from the at least one active agent reservoir to the biological interface.

In another aspect, the present disclosure is directed to a method of making an active agent laminate for an iontophoretic drug delivery device that provides transdermal delivery of one or more therapeutic active agents to a biological interface. The method includes preparing a lipophilic composition comprising a first surfactant and a nonpolar solvent, and preparing a hydrophilic composition comprising a second surfactant and a polar agent. The method further includes mixing the lipophilic composition and the hydrophilic composition using a high-shear mixer to form a pharmaceutically acceptable vehicle having a lipophilic phase and a hydrophilic phase. The method further includes impregnating at least one substrate with the pharmaceutically acceptable vehicle. The method further includes forming a multi-layer active agent laminate including the at least one substrate with the pharmaceutically acceptable vehicle and at least one delivery rate controlling membrane, and physically coupling the multi-layer active agent laminate to an active electrode assembly of an iontophoretic drug delivery device. In some embodiments, the active electrode assembly includes at least one active electrode element operable to provide an electromotive force to drive at least some of the pharmaceutically acceptable vehicle from the multi-layer active agent laminate to a biological interface.

In another aspect, the present disclosure is directed to a method for transdermal administration of at least one analgesic or anesthetic by iontophoresis. The method includes positioning an active electrode assembly and a counter electrode assembly of an iontophoretic delivery device on a biological interface of a subject. In some embodiments, the active electrode includes an active agent reservoir comprising at least one analgesic or anesthetic active agent carried by a pharmaceutically acceptable vehicle comprising at least one surfactant, at least one nonpolar solvent, and at least one polar agent.

The method further includes applying a sufficient amount of current to transport the at least one analgesic or anesthetic active agent from the active agent reservoir, to the biological interface of the subject, and to administer a therapeutically effective amount of the at least one analgesic or anesthetic active agent to produce analgesic or anesthetic therapy in the subject for a limited period of time.

In another aspect, the present disclosure is directed to an iontophoretic drug delivery device for providing transdermal delivery of one or more therapeutic active agents to a biological interface. The iontophoretic drug delivery device includes an active electrode assembly including at least one active electrode element and at least one active agent reservoir.

The at least one active agent reservoir includes a pharmaceutically acceptable vehicle comprising a plurality of first vesicles. In some embodiments, the first vesicles are selected from liposomes, nisomes, ethasomes, transfersomes, virosomes, cyclic oligosaccharides, non-ionic surfactant vesicles, and phospholipid surfactant vesicles. In some embodiments, at least some of the first vesicles include one or more therapeutic active agents.

The at least one active electrode element of the iontophoretic drug delivery device is operable to provide an electromotive force to drive at least some of the pharmaceutically acceptable vehicle including the plurality of first vesicles, from the at least one active agent reservoir to the biological interface.

In yet another aspect, the present disclosure is directed to an article of manufacture for transdermal administration of medication by iontophoresis. The article of manufacture includes an iontophoretic drug delivery device, at least one dosage form, and a package insert.

The iontophoretic drug delivery device includes an active electrode assembly including at least one active electrode element and at least one active agent reservoir. The at least one active agent reservoir includes a pharmaceutically acceptable vehicle including at least one surfactant, at least one nonpolar solvent, and at least one polar agent. In some embodiments, the at least one active electrode element is operable to provide an electromotive force to drive one or more active agents from the at least one active agent reservoir.

In some embodiments, the at least one dosage form includes one or more active agents selected from analgesics, anesthetics, or combinations thereof, and is loaded in the pharmaceutically acceptable vehicle.

The package insert provides instructions for transdermally administering, to a subject in need of pain therapy, a therapeutically effective amount of the at least one dosage form.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is a top, front view of a transdermal drug delivery system according to one illustrated embodiment.

FIG. 1B is a top, plan view of a transdermal drug delivery system according to one illustrated embodiment.

FIG. 2A is a schematic diagram of the iontophoresis device of FIGS. 1A and 1B comprising an active and counter electrode assemblies according to one illustrated embodiment.

FIG. 2B is a schematic diagram of the iontophoresis device of FIG. 2A positioned on a biological interface, with an optional outer release liner removed to expose the active agent, according to another illustrated embodiment.

FIG. 2C is a schematic diagram of the iontophoresis device comprising an active and counter electrode assemblies and a plurality of microneedles according to one illustrated embodiment.

FIG. 3A is a bottom, front view of a plurality of microneedles in the form of an array according to one illustrated embodiment.

FIG. 3B is a bottom, front view of a plurality of microneedles in the form of one or more arrays according to another illustrated embodiment.

FIG. 4 is a flow diagram of a method of transdermal administration of at least one analgesic or anesthetic by iontophoresis according to one illustrated embodiment.

FIG. 5 is a flow diagram of a method of making an active agent laminate for an iontophoretic drug delivery device that provides transdermal delivery of one or more therapeutic active agents to a biological interface according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with iontophoresis devices including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment” means that a particular referent feature, structure, or characteristic described in connection with the embodiment is included in at least one embodiment. Thus, the appearance of the phrases “in one embodiment,” or “in an embodiment,” or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an iontophoresis device including “an electrode element” includes a single electrode element, or two or more electrode elements. It should also be noted that the term “or” is generally employed in its sense including “and/or” unless the content clearly dictates otherwise.

As used herein the term “membrane” means a boundary, a layer, barrier, or material, which may, or may not be permeable. The term “membrane” may further refer to an interface. Unless specified otherwise, membranes may take the form a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane, for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH, and ACS also from Tokuyama Co., Ltd.

As used herein and in the claims, the term “bipolar membrane” means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules at a first rate, and some other molecules at a second rate different than the first. In yet further embodiments, the “semi-permeable membrane” may take the form of a selectively permeable membrane allowing only certain selective molecules to pass through it.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles). In some embodiments, a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

As used herein and in the claims, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

As used herein and in the claims, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., a drug, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent.

In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, ionizeable, and/or neutral therapeutic drug and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. Other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH.

The term “active agent” may also refer to electrically neutral agents, molecules, or compounds capable of being delivered via electro-osmotic flow. The electrically neutral agents are typically carried by the flow of, for example, a solvent during electrophoresis. Selection of the suitable active agents is therefore within the knowledge of one skilled in the relevant art.

In some embodiments, one or more active agents may be selected from analgesics, anesthetics, anesthetics vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of such active agents include lidocaine, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptamine 1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of anesthetic active agents or pain killers include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (Novocaine®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

As used herein and in the claims, the term “subject” generally refers to any host, animal, vertebrate, or invertebrate, and includes fish, mammals, amphibians, reptiles, birds, and particularly humans.

As used herein and in the claims, the term “agonist” refers to a compound that can combine with a receptor (e.g., a Toll-like receptor, and the like) to produce a cellular response. An agonist may be a ligand that directly binds to the receptor. Alternatively, an agonist may combine with a receptor indirectly by forming a complex with another molecule that directly binds the receptor, or otherwise resulting in the modification of a compound so that it directly binds to the receptor.

As used herein and in the claims, the term “antagonist” refers to a compound that can combine with a receptor (e.g., a Toll-like receptor, and the like) to inhibit a cellular response. An antagonist may be a ligand that directly binds to the receptor. Alternatively, an antagonist may combine with a receptor indirectly by forming a complex with another molecule that directly binds to the receptor, or otherwise results in the modification of a compound so that it directly binds to the receptor.

As used herein and in the claims, the term “effective amount” or “therapeutically effective amount” includes an amount effective at dosages and for periods of time necessary, to achieve the desired result. The effective amount of a composition containing a pharmaceutical agent may vary according to factors such as the disease state, age, gender, and weight of the subject.

As used herein and in the claims, the term “analgesic” refers to an agent that lessens, alleviates, reduces, relieves, or extinguishes a neural sensation in an area of a subject's body. In some embodiments, the neural sensation relates to pain, in other aspects the neural sensation relates to discomfort, itching, burning, irritation, tingling, “crawling,” tension, temperature fluctuations (such as fever), inflammation, aching, or other neural sensations.

As used herein and in the claims, the term “anesthetic” refers to an agent that produces a reversible loss of sensation in an area of a subject's body. In some embodiments, the anesthetic is considered to be a “local anesthetic” in that it produces a loss of sensation only in one particular area of a subject's body.

As one skilled in the relevant art would recognize, some agents may act as both an analgesic and an anesthetic, depending on the circumstances and other variables including but not limited to dosage, method of delivery, medical condition or treatment, and an individual subject's genetic makeup. Additionally, agents that are typically used for other purposes may possess local anesthetic or membrane stabilizing properties under certain circumstances or under particular conditions.

As used herein and in the claims, the term “immunogen” refers to any agent that elicits an immune response. Examples of an immunogen include, but are not limited to natural or synthetic (including modified) peptides, proteins, lipids, oligonucleotides (RNA, DNA, etc.), chemicals, or other agents.

As used herein and in the claims, the term “allergen” refers to any agent that elicits an allergic response. Some examples of allergens include but are not limited to chemicals and plants, drugs (such as antibiotics, serums), foods (such as milk, wheat, eggs, etc), bacteria, viruses, other parasites, inhalants (dust, pollen, perfume, smoke), and/or physical agents (heat, light, friction, radiation). As used herein, an allergen may be an immunogen.

As used herein and in the claims, the term “adjuvant” and any derivations thereof, refers to an agent that modifies the effect of another agent while having few, if any, direct effect when given by itself. For example, an adjuvant may increase the potency or efficacy of a pharmaceutical, or an adjuvant may alter or affect an immune response.

As used herein and in the claims, the terms “vehicle,” “carrier,” “pharmaceutically vehicle,” “pharmaceutically carrier,” “pharmaceutically acceptable vehicle,” or “pharmaceutically acceptable carrier” may be used interchangeably, and refer to pharmaceutically acceptable solid or liquid, diluting or encapsulating, filling or carrying agents, which are usually employed in pharmaceutical industry for making pharmaceutical compositions. Examples of vehicles include any liquid, gel, salve, cream, solvent, diluent, fluid ointment base, vesicle, liposomes, nisomes, ethasomes, transfersomes, virosomes, cyclic oligosaccharides, non ionic surfactant vesicles, phospholipid surfactant vesicles, micelle, and the like, that is suitable for use in contacting a subject.

In some embodiments, the pharmaceutical vehicle may refer to a composition that includes and/or delivers a pharmacologically active agent, but is generally considered to be otherwise pharmacologically inactive. In some other embodiments, the pharmaceutical vehicle may have some therapeutic effect when applied to a site such as a mucous membrane or skin, by providing, for example, protection to the site of application from conditions such as injury, further injury, or exposure to elements. Accordingly, in some embodiments, the pharmaceutical vehicle may be used for protection without a pharmacological agent in the formulation.

As used herein and in the claims, the term “cyclodextrin” refers to any of a family of cyclic oligosaccharides. Cyclodextrins, also sometimes called cycloamyloses, are composed of, but are not necessarily limited to, five or more D-glucopyranoside units, connected by α-(1,4) glycosidic linkages, as in amylose. Cyclodextrins having as many as 32 1,4-glucopyranoside units have been well characterized. Cyclic oligosaccharides as large as 150 units have been identified. Typically, cyclodextrins contain, but are not necessarily limited to, six to eight glucopyranoside units in a ring, commonly termed α-cyclodextrin (six units), β-cyclodextrin (seven units), and γ-cyclodextrin (eight units). These may be naturally occurring or produced synthetically. Cyclodextrins may be produced from starch by use of readily available enzymes, for example, α-amylase and cyclodextrin glycosyltransferase (CGTase), an enzyme that is produced by a number of different organisms. In certain methods known in the art, for example, starch may first be either heated or treated with α-amylase, followed by enzymatic conversion with CGTase. The conversion typically yields a mixture of the three common cyclodextrins, the ratio of which depends on the particular CGTase employed in the conversion reaction. The characteristic solubility of each of the three cyclodextrins in water is utilized in purification schemes. β-Cyclodextrin, for example, is poorly soluble in water, and may be isolated by crystallization. α- and γ-Cyclodextrins, which are much more water-soluble, may be purified chromatographically. Alternatively, synthetic methods utilizing certain organic agents may preferentially drive the reaction toward the formation of a specific cyclodextrin, by complexing with the specific cyclodextrin and causing it to precipitate from the reaction mixture as the conversion reaction proceeds. The specific cyclodextrin can then be isolated by recovery of the precipitate and separation from the agent used to form the complex.

The most stable three-dimensional configuration of a cyclodextrin is represented topologically as a toroid, wherein the smaller and the larger openings of the toroid expose primary and secondary hydroxyl groups, respectively, to the aqueous environment into which the cyclodextrin is placed. These regions are considerably less hydrophilic than the aqueous environment. The interior of the toroid is hydrophobic. The exterior of the toroidal cyclodextrin is sufficiently hydrophilic to allow it to dissolve in water.

Cyclodextrins are used in a broad range of applications in the pharmaceutical, food, and chemical industries. Complexes with a variety of relatively hydrophobic chemical substances may be formed in the apolar interior environment of the cyclodextrin cavity, resulting from a combination of van der Waals forces, hydrogen bonding, and hydrophobic interactions. Inclusion of a compound in the interior of a cyclodextrin may greatly modify the physical and/or chemical properties of that compound in solution. For example, inclusion of a relatively hydrophobic, poorly soluble pharmaceutical active agent within a cyclodextrin may enable such an agent to penetrate biological interfaces or body tissues by virtue of its increased compatibility with the aqueous environment. Having passed through a biological interface and/or into a body tissue, the decrease in concentration of the cyclodextrin complex in the aqueous environment may lead to spontaneous dissociation of the cyclodextrin, releasing the agent into the tissue. The rate of release may depend on the compatibility of the agent with the aqueous environment within the tissue. Alternatively, degradation of the complex to release the agent may take place as a result of specific conditions within the tissue. For example, controlled dissociation may result from a change in pH of the environment or from enzymatic action within the tissue. Once released into the aqueous environment within the tissue, the agent may exist either in solution or as a precipitate, depending for example on the solubility and concentration of the agent, as well as the concentration of any remaining cyclodextrin.

The headings provided herein are for convenience only and do not interpret the scope or meaning of the embodiments.

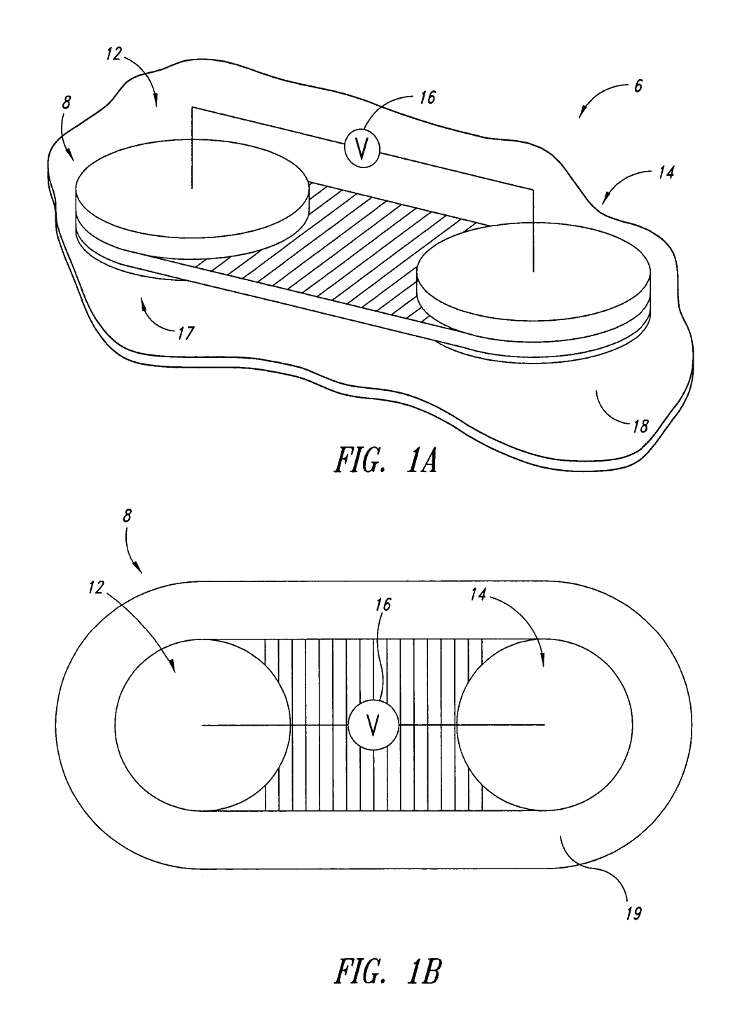

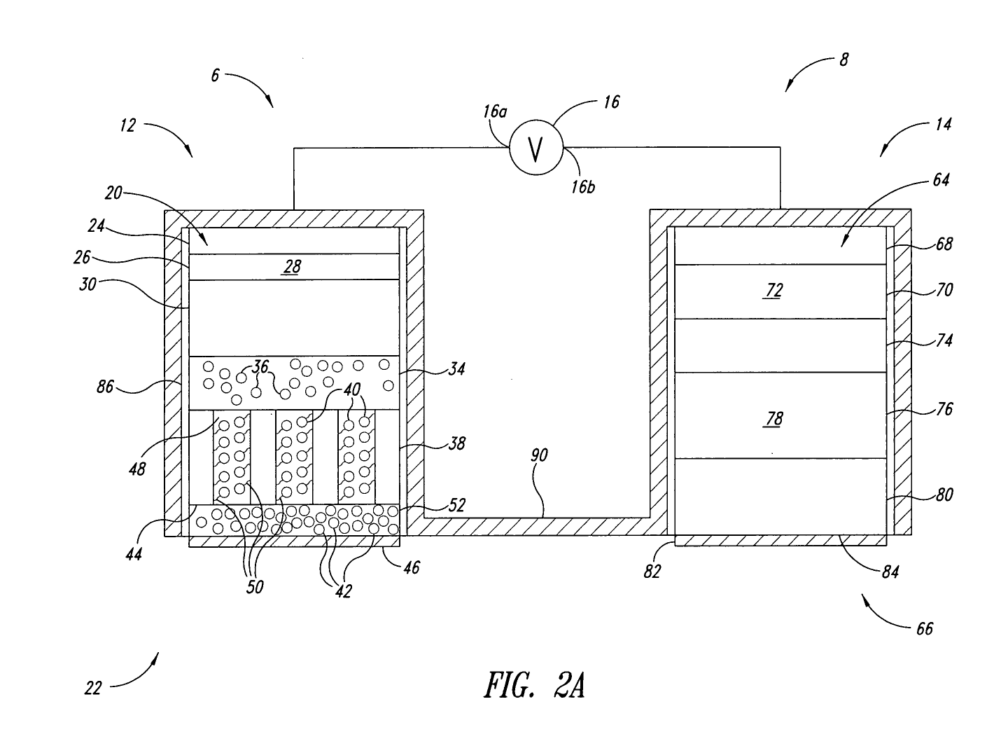

FIGS. 1A and 1B show an exemplary transdermal drug delivery system 6 for delivering of one or more active agents to a subject. The system 6 includes an iontophoresis device 8 including active and counter electrode assemblies 12, 14, respectively, and a power source 16. The active and counter electrode assemblies 12, 14, are electrically coupleable to the power source 16 to supply an active agent contained in the active electrode assembly 12, via iontophoresis, to a biological interface 18 (e.g., a portion of skin or mucous membrane). In some embodiments, the iontophoresis device 8 may optionally include an outer adhesive surface 19 for physically coupling the iontophoresis device 8 to the biological interface 18 of the subject.

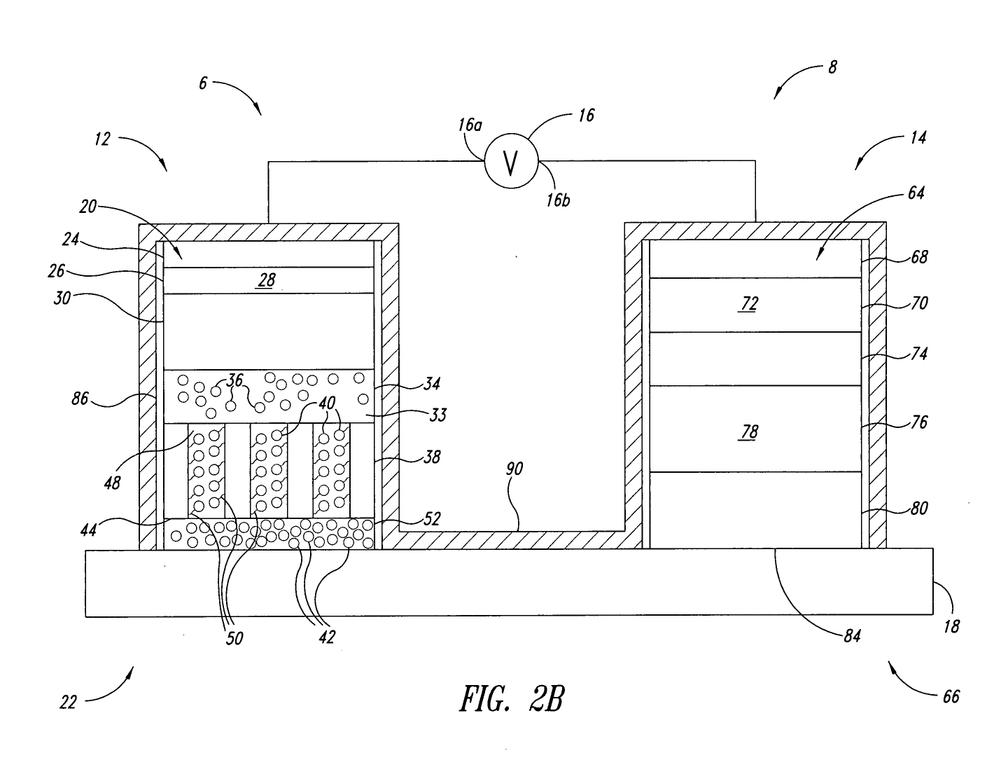

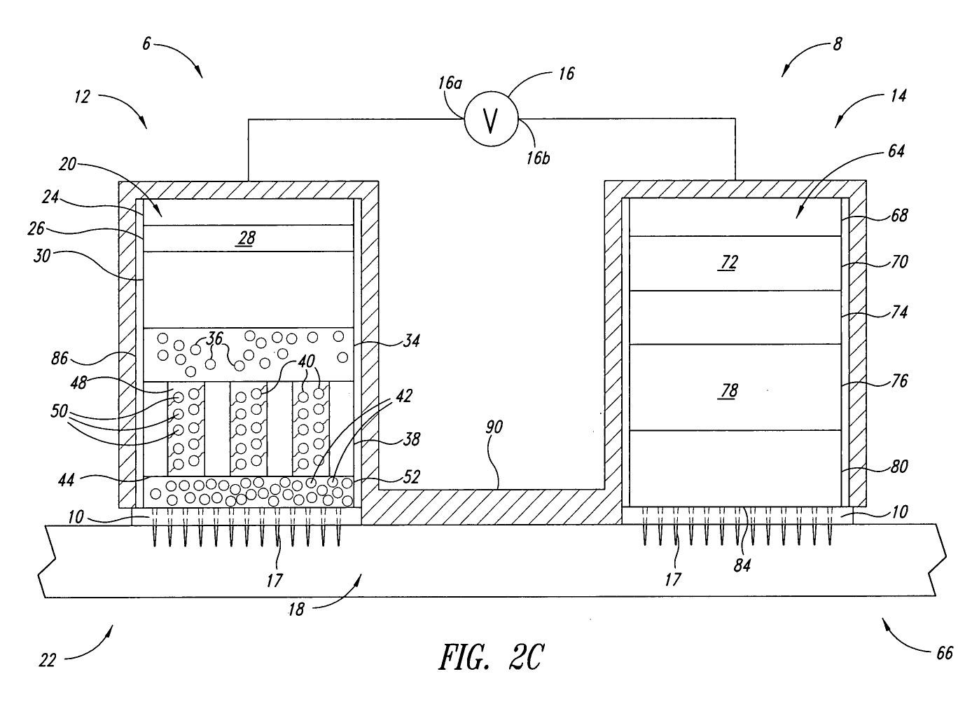

As shown in FIGS. 2A and 2B, the active electrode assembly 12 may further comprise, from an interior 20 to an exterior 22 of the active electrode assembly 12: an active electrode element 24, an electrolyte reservoir 26 storing an electrolyte 28, an inner ion selective membrane 30, one or more inner active agent reservoirs 34, storing one or more active agents 36, an optional outermost ion selective membrane 38 that optionally caches additional active agents 40, and an optional further active agent 42 carried by an outer surface 44 of the outermost ion selective membrane 38. The active electrode assembly 12 may further comprise an optional outer release liner 46.

The one or more active agent reservoirs 34 are loadable with a vehicle for transporting, delivering, encapsulating, and/or carrying the one or more active agents 36, 40, 42.

Examples of vehicles include degradable or non-degradable polymers, hydrogels, organogels, liposomes, nisomes, ethasomes, transfersomes, virosomes, cyclic oligosaccharides, non-ionic surfactant vesicles, phospholipid surfactant vesicles, micelles, microspheres, creams, emulsions, lotions, pastes, gels, ointments, organogel, and the like, as well as any matrix that allows for transport of an agent across the skin or mucous membranes of a subject. In at least one embodiment, the vehicle allows for controlled release formulations of the compositions disclosed herein.

As one skilled in the relevant art would appreciate, pharmaceutical formulations employed in forming, for example, pharmaceutically acceptable vehicles for transporting one or more active agents 36, 40, 42 will be readily understood in the art. For example, ointments may be semisolid preparations based on petrolatum or other petroleum derivatives. Emulsions may be water in oil or oil in water and include, for example, cetyl alcohol, gylceryl monostearate, lanolin and steric acid, and may also contain polyethylene glycols. Creams may be viscous liquids or semisolid emulsions of oil in water or water in oil. Gels may be semisolid suspensions of molecules including organic macromolecules as well as an aqueous, alcohol, and/or oil phase. Examples of such organic macromolecules include gelling agents (e.g., carboxypolyalkylenes, and the like), hydrophilic polymers (e.g., polyethylene oxides, polyoxyethylene-polyoxypropylene copolymers, polyvinylalcohols, and the like) cellulosic polymers (e.g., hydroxypropyl cellulose, hydroxyethyl cellulose, hydroxypropyl methylcellulose, hydroxypropyl methylcellulose, phthalate, methyl cellulose, and the like), tragacanth or xanthan gums, sodium alginate, gelatin, and the like, or combination thereof.

In some embodiments, the pharmaceutically acceptable vehicle includes at least one surfactant, at least one nonpolar solvent, and at least one polar agent.

The at least one surfactant may act as a gelling and/or viscosifier substance (i.e., a gelator, a thickener, a gellant molecule, a gelling agent). The at least one surfactant may be available from naturally occurring sources or synthetically made by techniques well known in the art. For example, lecithin is a naturally occurring mixture of the diglycerides of stearic, palmitic, and oleic acids, linked to the choline ester of phosphoric acid, commonly called phosphatidylcholine. Hydrogenated lecithin, however, is the product of controlled hydrogenation of lecithin. Further examples of suitable surfactants in the form of phospholipids from naturally occurring sources include sphingolipids including sphingosine and derivatives (from soybean, egg, brain & milk), gangliosides, phytosphingosine and derivatives (from yeast), phosphotidylethanolamine, phosphotidylserine, and phosphotydylinositol.

Pharmaceutically, lecithins are mainly used as dispersing, emulsifying, and stabilizing agents and are typically included in intramuscular (IM) and intravenous (IV) injections, parenteral nutritional formulations, and topical products. Lecithin is also listed in the FDA Inactive Ingredients Guide for use in inhalations, IM and IV injections, oral capsules, suspensions and tablets, rectal, topical, and vaginal preparations.

Other suitable examples of the at least one surfactant include one or more emulsifying agents, amphoteric surfactants, non-ionic surfactants, ionic surfactants, acetone-insoluble phosphatides, phospholipids, amphiphiles, biocompatible surfactants, ether lipids, fluoro-lipids, polyhydroxyl lipids, polymerized liposomes, lecithin, hydrogenated lecithin, naturally occurring lecithin, egg lecithin, hydrogenated egg lecithin, soy lecithin, hydrogenated soy lecithin, vegetable lecithin, sorbitan esters, sorbitant monoesters, sorbitan monolaurate, sorbitan monooleate, sorbitan monopalmitate, sorbitan monostearate, sorbitan monostearate-palmitate, sorbitan sexquioleate, sorbitan tristearate, sorbitan trioleate, diacylglycerols, gangliosides, glycerophospholipids, lysophospholipids, mixed-chain phospholipids, pegylated phospholipids, phosphatidic acids, phosphatidylcholines, phosphatidylethanolamines, phosphatidylinositols, phosphocholines, phosphoethanolamines, phosphoglycerols, phosphoserines, phytosphingosines, poloxamers, polyoxypropylene-polyoxethylene block copolymers, sphingosines, and the like, or combinations thereof.

In some embodiments, the at least one surfactant is selected from surfactants having at least a glycerol residue moiety, one or more C₁₀-C₂₀ hydrocarbon chain moieties, and a moiety selected from phosphatidic acids, phosphatidylcholines, lyso-phosphatidylcholines, phosphatidylethanol amines, lysol-phosphatidylethanol amines, phosphatidylserines, phosphatidylinositols, and the like, or other groups cable of forming a hydrogen-bonding network. The C₁₀-C₂₀ hydrocarbon chain moieties may include saturated or unsaturated, linear or branched, substituted or unsubstituted alkyl groups. In some further embodiments, the at least one surfactant further includes a choline head group. Examples of such surfactants are well known in the art and include, for example, lecithin, phosphocholines, and the like. See, for example, Kumar et aL, “Lecithin Organogels as a Potential Phospholipid-Structured System for Topical Drug Delivery: A Review” AAPS PharmSciTech 6(2) Article 40 (2005).

In some embodiments, the at least one surfactant is selected from amphiphilic phospholipids including, for example, phosphatidylcholine. Amphiphilic phospholipids contain both hydrophobic and hydrophilic groups are major constituents of, for example, cell membranes. The amphiphilic phospholipids are capable of forming a phospholipid bilayer with their hydrophilic (polar) heads facing their aqueous surroundings (e.g., the cytosol) and their hydrophobic tails facing each other.

The pharmaceutically acceptable vehicle for transporting the one or more active agents 36, 40, 42 may include at least a first surfactant and a second surfactant. In some embodiments, the first surfactant may be selected from phosphatidylcholines, and the second surfactants may be selected from poloxamers and polyoxypropylene-polyoxethylene block copolymers. In some other embodiments, the first surfactant is selected from lecithin, hydrogenated lecithin, naturally occurring lecithin, egg lecithin, hydrogenated egg lecithin, soy lecithin, hydrogenated soy lecithin, and vegetable lecithin, and the second surfactant is selected from poloxamers and polyoxypropylene-polyoxethylene block copolymers.

In some embodiments, the pharmaceutically acceptable vehicle further includes at least one nonpolar solvent. Examples of suitable nonpolar solvents include organic solvents, vegetable oils; saturated or unsaturated, linear or branched, substituted or unsubstituted, alkanes; ethers; esters; fatty acids; amines; and the like. In some embodiments, the nonpolar solvent is selected from ethyl laureate, ethyl myristate, isopropyl myristate, isopropyl palmitate, cyclopentane, cyclooctane, trans-decalin, trans-pinane, n-pentane, n-hexane, n-hexadecane, tripropylamine, and the like.

In some embodiments, the pharmaceutically acceptable vehicle further includes at least one polar agent. Examples of polar agents include alcohols, polyalcohols, glycerol, glycerols, polyglycerols, ethylene glycols polyglycols, formamide, water, and the like, or combinations thereof. The at least one polar agent component of the pharmaceutically acceptable vehicle may acts as a structure forming and stabilizing agent. In some embodiments, the polar agent is partially responsible for the formation of tubular or cylindrical micellar aggregates that form part of a matrix of entangled reverse tubular or cylindrical micelles. In some embodiments, water is employed as the polar agent.

In some embodiments, the pharmaceutically acceptable vehicle further includes a complex of a cyclodextrin with at least one active agent.

The pharmaceutically acceptable vehicle may take a variety of forms including a colloidal dispersion having an aqueous phase and a lipid phase, a gel, an organogel, a lecithin organogel, a pluronic lecithin organogel, and the like, or combinations thereof. In at least one embodiment, the pharmaceutically acceptable vehicle takes the form of an organogel. In at least one embodiment, the pharmaceutically acceptable vehicle takes the form of a lecithin organogel. Lecithin formed organogels are typically clear, thermodynamically stable, and biocompatible. They may also be non-polymeric, viscoelastic, and isotropic in form.

The pharmaceutically acceptable vehicle may further include at least one additional surfactant selected from synthetic polymers, for example, pluronics. The term “pluronics,” sometimes referred to as poloxamers, poloxamer polyol, and lutrols, is usually descriptive of a series of nonionic, closely related block copolymers of, for example, ethylene oxide and/or propylene oxide. Pluronics may be useful as co-surfactants, emulsifiers, solubilizers, suspending agents and/or stabilizers. In some embodiments, the pharmaceutically acceptable vehicle takes the form of a pluronic lecithin organogel. In some other embodiments, the pharmaceutically acceptable vehicle takes the form of a pluronic lecithin organogel that includes at least one lecithin surfactant and at least one pluronic co-surfactant.

Examples of pluronic surfactants include 1,2-Propyleneglycol (ethoxylated and propoxylated), 106392-12-5, 11104-97-5, 53637-25-5, 60407-69-4, 65187-10-2, 69070-95-7, 75-H-1400, 75H90000, 9003-11-6, Adeka 25R1, Adeka 25R2, Adeka L 61, Adeka Pluronic F 108, AIDS162017, AIDS-162017, Antarox 17R4, Antarox 25R2, Antarox B 25, Antarox F 108, Antarox F 68, Antarox F 88, Antarox F 88FL, Antarox L 61, Antarox L 72, Antarox P 104, Antarox P 84, Antarox SC 138, Arco Polyol R 2633, Arcol E 351, B 053, BASF-L 101, Berol TVM 370, Bloat guard, Block polyethylene-polypropylene glycol, Block polyoxyethylene-polyoxypropylene, Breox BL 19-10, BSP 5000, C13430, Cirrasol ALN-WS, Crisvon Assistor SD 14, CRL 1005, CRL 1605, CRL 8131, CRL 8142, D 500 (polyglycol), Daltocel F 460, Dehypon KE 3557, Detalan, Eban 710, Emkalyx EP 64, Emkalyx L 101, Emkalyx L101, Empilan P 7068, Emulgen PP 230, Epan 450, Epan 485, Epan 710, Epan 750, Epan 785, Epan U 108, Epon 420, Ethylene glycol-propylene glycol block copolymer, Ethylene glycol-propylene glycol polymer, Ethylene oxide-propylene oxide block polyemr, Ethylene oxide-propylene oxide block copolymer dipropylene glycol ether, Ethylene oxide-propylene oxide block copolymer ether with ethylene glycol, Ethylene oxide-propylene oxide block polymer, Ethylene oxide-propylene oxide copolymer, F 108, F 127, F 77, F 87, F 88, F-108, Genapol PF 10, Polyethylenepolypropylene Glycols, Polyethylene-polypropylene Glycols, HSDB 7222, Hydrowet, Laprol 1502, LG 56, Lutrol F, Lutrol F (TN), M 90/20, Magcyl, Meroxapol 105, Methyloxirane polymer with oxirane block, Methyloxirane-oxirane copolymer, Methyloxirane-oxirane polymer, Monolan 8000E80, Monolan PB, N 480, Newpol PE-88, Niax 1646, Niax LG 56, Nissan Pronon 201, Nixolen SL 19, NSC 63908, NSC63908, Oligoether L-1502-2-30, P 103, P 104, P 105, P 123, P 65, P 84, P 85, PEG/PPG-125/30 Copolymer, Plonon 201, Plonon 204, Pluracare, Pluracol V, Pluriol PE, Pluriol PE 6810, Pluronic, Pluronic 10R8, Pluronic 31R2, Pluronic C 121, Pluronic F, Pluronic F 108, Pluronic F 125, Pluronic F 127, Pluronic F 38, Pluronic F 68, Pluronic F 68LF, Pluronic F 87, Pluronic F 88, Pluronic F 98, Pluronic F108, Pluronic F127, Pluronic F68, Pluronic F-68, Pluronic F77, Pluronic F86, Pluronic F87, Pluronic F87-A7850, Pluronic F88, Pluronic L, Pluronic L 101, Pluronic L 121, Pluronic L 122, Pluronic L 24, Pluronic L 31, Pluronic L 35, Pluronic L 44, Pluronic L 61, Pluronic L 62, Pluronic L 64, Pluronic L 68, Pluronic L 92, Pluronic L-101, Pluronic 144, Pluronic L62, Pluronic 162 (mw 2500), Pluronic L64, Pluronic I64 (mw 2900), Pluronic L-81, Pluronic P, Pluronic P 104, Pluronic P 75, Pluronic P 85, Pluronic P103, Pluronic P104, Pluronic P105, Pluronic P123, Pluronic P65, Pluronic P-65, Pluronic P-75, Pluronic P84, Pluronic P85, Pluronic-68, Poloxalene, Poloxalene [USAN:BAN:INN], Poloxalene L64, Poloxalkol, Poloxamer, Poloxamer [USAN:BAN:INN], Poloxamer 101, Poloxamer 108, Poloxamer 182LF, Poloxamer 188, Poloxamer 331, Poloxamer 407, Poloxamer-188, Poly (propylene oxide-ethylene oxide), Poly(ethylene oxide-co-propylene oxide), Poly(mixed ethylene and propylene) glycol, Poly(oxyethylene)-poly(oxypropylene) glycol, Poly(oxyethylene)-poly(oxypropylene) polymer, Polyethylene glycol, propoxylated, Polyethylene oxide-polypropylene oxide, Polyethylene oxide-polypropylene oxide copolymer, Polyethylene-Pluronic L-62LF, Polyethylene-polypropylene glycol, Polykol, Polylon 13-5, Polyoxamer 108, Polyoxyethylenated poly (oxypropylene), Polyoxyethylene-polyoxypropylene block copolymer, Polyoxyethylene-polyoxypropylene copolymer, Polyoxyethylene polyoxypropylene, Polyoxyethylene-oxy-propylene [French], Polyoxyethylene-polyoxypropylene, Polyoxyethylene-polyoxypropylene polymer, Polyoxypropylene-polyoxyethylene block copolymer, Polypropoxylated, polyethoxylated propylene glycol, Polypropylene glycol, ethoxylated, Polypropylene glycol-ethylene oxide copolymer, PPG Diol 3000EO, Proksanol, Pronon, Pronon 102, Pronon 104, Pronon 201, Pronon 204, Pronon 208, Propane-1,2-diol,ethoxylated propoxylated, Propylen M 12, Propylene glycol-propylene oxide-ethylene oxide polymer, Propylene oxide-ethylene oxide copolymer, Propylene oxide-ethylene oxide polymer, Proxanol, Proxanol 158, Proxanol 228, Proxanol Tsl-3, RC 102, Regulaid, Rokopol 16P, Rokopol 30P, Rokopol 30P9, SK and F 18,667, SK&F 18,667, SKandF 18,667, Slovanik, Slovanik 630, Slovanik 660, Slovanik M-640, Supronic B 75, Supronic E 400, Synperonic PE 30/40, Tergitol monionic XH, Tergitol nonionic XH, Tergitol XH, Tergitol XH (nonionic), Teric PE 61, Teric PE 62, Teric PE40, Teric PE60, Teric PE70, Thanol E 4003, Therabloat, TsL 431, TVM 370, Unilube 50MB168X, Unilube 50MB26X, Velvetol OE 2NT1, Voranol P 2001, WS 661, Wyandotte 7135, α-Hydro-Ω-hydroxypoly(oxyethylene)ₐ-poly(oxopropylene)_{b}-poly(oxye thylene)ₐ block copolymer, α-Hydro-Ω-hydroxypoly(oxyethylene)ₐ-poly(oxopropylene)_{b} poly(oxyethylene)ₐ block copolymer, α-Hydro-Ω-hydroxypoly(oxyethylene) poly(oxypropylene) poly(oxyethylene) block copolymer, and the like.

Protocols for forming pharmaceutically acceptable vehicle in the form of gels are well known in the art. For example, various forms of lecithin organogels are described in Kumar et aL., “Lecithin Organogels as a Potential Phospholipid-Structured System for Topical Drug Delivery: A Review” AAPS PharmSciTech 6(2) Article 40 (2005).

The stability and mechanical properties of pharmaceutical vehicles, in the form of organogels, in combination with iontophoresis, may provide for novel and more effective methods for transdermal or transmucosal delivery of the one or more agents 36, 40, 42. For example, in some embodiments, the transdermal or transmucosal penetration efficiency of a pharmaceutically acceptable vehicle for transporting one or more active agents 36, 40, 42 may be greatly enhanced by employing an electromotive force and/or current to transfer the pharmaceutically acceptable vehicle comprising the one or more active agents 36, 40, 42, and loaded in the at least one active agent reservoir 34 of an iontophoresis delivery device 8, to the biological interface 18.

The resulting pharmaceutically acceptable vehicles in the form of organogels may include an aqueous phase and an organic phase. In some embodiments, the pharmaceutically acceptable vehicle may have a dispersed phase and a continuous phase. The organic phase may include phospholipids that may aid in crossing the epidermis or other mucous membranes of the subject, thereby facilitating pharmaceutical delivery of the one or more active agents 36, 40, 42. In some embodiments, the aqueous phase may include one or more active agents selected from hydrophilic active agents and charged hydrophilic active agents. In some embodiments, the organic phase may include one or more active agents selected from amphiphilic active agents, and lipophilic active agents. Is some other embodiments, the aqueous phase may include one or more active agents selected from amphiphilic active agents, hydrophilic active agents and charged hydrophilic active agents, and the organic phase may include one or more active agents selected from amphiphilic active agents, and lipophilic active agents.

In some embodiments, the pharmaceutically acceptable vehicle includes at least a first surfactant and a second surfactant, at least one nonpolar solvent, and at least one polar agent. The first surfactant is selected from lecithin, hydrogenated lecithin, naturally occurring lecithin, egg lecithin, hydrogenated egg lecithin, soy lecithin, hydrogenated soy lecithin, and vegetable lecithin, and the second surfactant is selected from poloxamers and polyoxypropylene-polyoxethylene block copolymers. The at least one nonpolar solvent is selected from ethyl laureate, ethyl myristate, isopropyl myristate, isopropyl palmitate, cyclopentane, cyclooctane, trans-decalin, trans-pinane, n-pentane, n-hexane, n-hexadecane, and tripropylamine; and the at least one polar agent is selected from water, alcohols, polyalcohols, glycerol, glycerols, polyglycerols, ethylene glycol, polyglycols, and formamide.

In some embodiments, the pharmaceutically acceptable vehicle may be formulated as a controlled-release or sustained-release formulation.

The pharmaceutically acceptable vehicle may further comprise a therapeutically effective amount of one or more active agents 36, 40, 42. In some embodiments, the one or more active agents 36, 40, 42 are selected from cationic, anionic, ionizable, or neutral active agents. In some embodiments, the pharmaceutically acceptable vehicle comprising one or more active agents 36, 40, 42 is loaded in the one or more active agent reservoirs 34, of the iontophoresis delivery device 8.

In some embodiments, the one or more active agents 36, 40, 42 may be capable of increasing, decreasing, altering, initiating, and/or extinguishing a biological response. As one skilled in the relevant art would recognize, dosing of a particular active agent may depend on the specific medical condition or indication, method of treatment or delivery, the subject's age, the subject's weight, the subject's gender, the subject's genetic makeup, the subject's overall health, as well as other factors. In some embodiments, the iontophoresis delivery device 8 may be configured to provide controlled-delivery or sustained-delivery of the pharmaceutically acceptable vehicle including one or more active agents 36, 40, 42.

Examples of the one or more active agents 36, 40, 42 include one or more immuno-adjuvants, immuno-modulators, immuno-response agents, immuno-stimulators, specific immuno-stimulators, non-specific immuno-stimulators, and immuno-suppressants, vaccines, agonists, antagonist, opioid agonist, opioid antagonist, antigens, adjuvants, immunological adjuvants, immunogens, tolerogens, allergens, toll-like receptor agonists, toll-like receptor antagonists, and the like, or combinations thereof.

Further examples of the at one or more active agents 36, 40, 42 include at least one analgesic or anesthetic active agent selected from alfentanil, codeine, COX-2 inhibitors, opiates, opioid agonist, opioid antagonist, diamorphine, fentanyl, meperidine, methadone, morphine morphinomimetics, naloxone, nonsteroidal anti-inflammatory drugs (NSAIDs), oxycodone, remifentanil, sufentanil, and tricyclic antidepressants, or combinations thereof. In some embodiments, the one or more active agents 36, 40, 42 are selected from analgesics, anesthetics, or combinations thereof.

As one skilled in the relevant art would recognize, multiple and various analgesics may be employed as active agents 36, 40, 42. Suitable analgesics include, for example, non-steroidal anti-inflammatory compounds, natural and synthetic opiates or opioids, morphine, Demorole (meperidine), Dilaudid® (hydromorphone), SublimazeS (fentanyl), acetaminophen, Darvocet® (propoxyphene and acetaminophen), codeine, naproxen, aspirin, ibuprofen, Vicodin® (hydrocodone bitartrate and acetaminophen), Percocet® (acetaminophen and oxycodone), Vicoprofen® (hydrocodone and ibuprofen), Ultram® (tramadol), Dolphine® (methadone), OxyContin® (oxycodone), COX-2 inhibitors (such as celecoxib and rofecoxib), prednisone, etodolac, nabumetone, indomethacin, sulindac, tolmetin sodium, ketorolac tromethamine, trisalicylate, diflunisal, salsalate, sodium salicylate, sodium thiosalicylate, flurbiprofen, fenoprofen, ketoprofen, oxaprozin, piroxicam, isoxicam, meclofenamate, diclofenac, epinephrine, benzodiazepines, cannabinoids, caffeine, hydroxyzine, and the like, or any combination thereof.

Some analgesics may function, for example, by interfering with nerve reception or response, by interfering with cell receptors, by interfering with production of a cellular component, by interfering with regulation of a particular gene transcription or protein translation, by interfering with protein excretion or secretion, by interfering with cellular membrane components, any combination thereof, or by other means. Some local anesthetics may cause reversible loss of sensation in an area of a subject's body by interrupting nerve impulses or responses, by influencing membrane variations, by influencing production of cellular components, by interrupting nerve conductance, by interrupting gene transcription or protein translation, by interfering with protein secretion or excretion, any combination thereof, or by other means. Some topical anesthetics may have a rapid onset of action (for example, approximately in 10 minutes or less, approximately in 5 minutes or less, etc.), and/or may have a moderate duration of action (approximately 30-60 minutes, or more).

As one skilled in the relevant art would recognize that multiple and various anesthetics could be employed. For example, several suitable local anesthetic agents consist of an aromatic ring linked by a carbonyl-containing moiety through a carbon chain to a substituted amino group, including esters, amides, quinolones, and the like. In certain embodiments, the anesthetic may be present in the composition as a free base to promote penetration of the agent through the skin or mucosal surface. Examples of some other anesthetics include centbucridine, tetracaine, Novocaine® (procaine), ambucaine, amolanone, amylcaine, benoxinate, betoxycaine, carticaine, chloroprocaine, cocaethylene, cyclomethycaine, butethamine, butoxycaine, carticaine, dibucaine, dimethisoquin, dimethocaine, diperodon, dyclonine, ecogonidine, ecognine, euprocin, fenalcomine, formocaine, hexylcaine, hydroxyteteracaine, leucinocaine, levoxadrol, metabutoxycaine, methyl chloride, myrtecaine, butamben, bupivicaine, mepivacaine, betα-adrenoceptor antagonists, opioid analgesics, butanilicaine, ethyl aminobenzoate, fomocine, hydroxyprocaine, isobutyl p-aminobenzoate, naepaine, octacaine, orthocaine, oxethazaine, parenthoxycaine, phenacine, phenol, piperocaine, polidocanol, pramoxine, prilocalne, propanocaine, proparacaine, propipocaine, pseudococaine, pyrrocaine, salicyl alcohol, parethyoxycaine, piridocaine, risocaine, tolycaine, trimecaine, tetracaine, anticonvulsants, antihistamines, articaine, cocaine, procaine, amethocaine, chloroprocaine, Lidocaine® (xylocaine), marcaine, chloroprocaine, etidocaine, prilocaine, lignocaine, benzocaine, zolamine, ropivacaine, dibucaine, and the like or pharmaceutically acceptable salt thereof, or mixtures thereof.

In some embodiments, the pharmaceutically acceptable vehicle may further comprise a therapeutically effective amount of one or more immunity agents. An immunity agent may be capable of increasing, decreasing, altering, initiating or extinguishing an immune response. As one skilled in the relevant art would recognize, dosing of a particular active agent may depend on the specific medical condition or indication, method of treatment or delivery, the subject's age, the subject's weight, the subject's gender, the subject's genetic makeup, the subject's overall health, as well as other factors. In at least one embodiment of the pharmaceutical composition, the immunity agent is capable of functioning as an adjuvant. In certain embodiments, the immunity agent is a Toll-like receptor agonist or antagonist.

Toll-like receptors may initiate immune responses by, among other things, activating dendritic cells. For example, some toll-like receptors belong to a family of receptors called pattern-recognition receptors, which may be activated upon recognition of “Pathogen-Associated Molecular Patterns” or PAMPs. PAMPs are molecular patterns common to many pathogens. Examples of some PAMPs include, but are not limited to, cell wall constituents such as lipopolysaccharide, peptidoglycan, lipoteichoic acid, lipoarabinomannan, single or double stranded RNA, and unmethylated CpG DNA.

A number of Toll-like receptors have been identified in mammals and are included in various embodiments of the present disclosure. For example, TLR1, TLR2, TLR3, TLR4, TLR5, TLR6, TLR7, TLR8, TLR9, TLR10, TLR11, TLR12 (mouse only), TLR13 (mouse only), have all been identified in mice and/or humans. Agonists or antagonists to any and/or all of these Toll-like receptors and others not yet identified may be included in various embodiments.

Stimulation of Toll-like receptors by pathogens results in expression of multiple immune response genes, including NF-KB, mitogen activated protein kinases p38, Jun-N-terminal kinase, and the interferon pathway.

Some examples of Toll-like receptor agonists include, but are not limited to, isatoribine, natural or synthetic lipopeptides (e.g., Pam3CSK4, also called palmitoyl-3-cysteine-serine-lysine4), bacteria or fragments of bacteria, including heat killed L. Monocytogenes (HLKM) and Flagellin S. typhimudum, natural or synthetic RNA (e.g., Poly (I:C) and ssRNA40), natural or synthetic lipopolysaccharides (e.g., LPS E. coli K12), natural or synthetic oligonucleotides or oligonucleotide analogues (e.g., imiquimod and ODN2006), and the like. Additionally, Toll-like receptor agonists that have not yet been identified may also be included in various embodiments.

Some examples of Toll-like receptor antagonists include, but are not limited to natural or synthetic lipopolysaccharides (e.g., LPS-PG, isolated from P. gingivalis; and LPS-EK msbB, isolated from E. coli K12 msbB), or natural or synthetic oligonucleotides (e.g., ODN 2088 (suppressive ODN, mouse specific); and ODN TTAGGG (suppressive ODN, human specific)), and the like. Additionally, Toll-like receptor antagonists that have not yet been identified may also be included in various embodiments.

One skilled in the relevant art would recognize that some or all of the compositions herein described are suitable for pharmaceutical compositions. At least some embodiments include a pharmaceutical composition comprising a pharmaceutically acceptable vehicle and an effective amount of an active agent in the form of an active immunity agent. In at least one embodiment of the pharmaceutical composition, the immunity agent is a Toll-like receptor agonist. In at least one embodiment of the pharmaceutical composition, the immunity agent is a Toll-like receptor antagonist. In at least one embodiment of the pharmaceutical composition, the vehicle allows for controlled release of the immunity agent.

In some embodiments, the pharmaceutically acceptable vehicle may further comprise an adjuvant. Multiple different adjuvants are known in the art, and are described, for example, in William E. Paul “Fundamental Immunology” Lippincot Williams & Wilkins (5th ed. 2003) and Janeway et al. “Immunobiology” Elsevier Science Health Science div (6th ed., 2004).

In some embodiments, the adjuvant alters the immune response of the biological factor administered in conjunction with the adjuvant. In at least one aspect, the adjuvant alters the potency of an immune response. In at least one aspect, the adjuvant alters the type of immune response to the biological factor. In at least one aspect, the adjuvant increases the potency of an immune response.

In at least one aspect, the adjuvant decreases the potency of an immune response. In at least one aspect, the adjuvant alters both the potency and the type of immune response to the biological factor. The biological factor may be injected, orally administered, iontophoretically administered or otherwise introduced to a subject.

As used herein and in the claims, “in conjunction with” and any derivations thereof, refers to administration of the adjuvant simultaneously with, prior to, or subsequent to administration of the biological factor. In at least one embodiment, the adjuvant is administered simultaneously with the biological factor. In at least one embodiment, the adjuvant is administered prior to the biological factor. In at least one embodiment, the adjuvant is administered subsequent to the biological factor.

Some adjuvants may alter an immune response to a biological factor administered in conjunction with the adjuvant, while not altering an immune response when the adjuvant is administered alone. Examples of adjuvants that may act directly or indirectly on an immune system or on hematopoeitic cells and/or components include antigen presenting cells, such as dendritic cells and Langerhans cells, and/or other components such as lymphocytes (T cells, B cells, etc.), monocytes, macrophages, neutrophils, eosinophils, red blood cells, platelets, basophils, and/or supportive cells (stromal cells, stem cells, tissue cells), or any combination thereof. In addition, an adjuvant may alter production or degradation of chemicals associated with immune responses, including cytokines, nitric oxide, heat shock proteins, vasodilators, vasoconstrictors, neurotransmitters, other neurotrophic factors, hemoglobin, and any other biological chemical that may affect an immune system component.

In some embodiments, the pharmaceutically acceptable vehicle may further comprise one or more additional ingredients, such as one or more thickening agents, medicinal agents, growth factors, immune system agents, wound-healing factors, peptidomimetics, proteins or peptides, carbohydrates, bioadhesive polymers, preservatives, inert carriers, caffeine or other stimulants (such as epinephrine, norepinephrine, adrenaline, etc.), lipid absorbents, chelating agents, buffers, anti-fading agents, stabilizers, moisture absorbents, vitamins, UV blockers, humectants, cleansers, colloidal meals, abrasives, herbal extracts, phytochemicals, fragrances, colorants or dyes, film-forming materials, analgesics, etc. A single excipient may perform multiple functions or a single function. One skilled in the relevant art will readily be able to identify and choose any such excipients based on the desired physical and chemical properties of the final formulation.

Examples of some commonly used thickening agents include, but are not limited to, cellulose, hydroxypropyl cellulose, methyl cellulose, polyethylene glycol, sodium carboxymethyl cellulose, polyethylene oxide, xanthan gum, guar gum, agar, carrageenan gum, gelatin, karaya, pectin, locust-bean gum, aliginic acid, bentonite carbomer, povidone, tragacanth, and the like, or any combination thereof.

One skilled in the relevant art would also readily be able to identify and choose any optional medicinal agents or their pharmaceutically acceptable salts, based on the desired effect for the final formulation. Examples of medicinal agents include, but are not limited to, antifungal compositions (e.g., ciclopirox, triacetin, nystatin, tolnaftate, miconizole, clortrimazole, and the like), antibiotics (gentamicin, polymyxin, bacitracin, erythromycin, and the like), antiseptics (iodine, povidine, benzoic acid, benzyol peroxide, hydrogen peroxide, and the like), and anti-inflammatory compositions (e.g., hydrocortisone, prednisone, dexamethasone, and the like), or any combination thereof.

One skilled in the relevant art would also readily identify and choose any optional bioadhesive polymers that may be useful for hydrating the skin, ensuring surface contact and/or increasing pharmaceutical delivery. Some examples of bioadhesive polymers include, but are not limited to pectin, alginic acid, chitosan, hyaluronic acid, polysorbates, polyethyleneglycol, oligosaccharides, polysaccharides, cellulose esters, cellulose ethers, modified cellulose polymers, polyether polymers and oligomers, polyether compounds (block copolymers of ethylene oxide and propylene oxide) polyacrylamide, poly vinyl pyrrolidone, polymethacrylic acid, polyacrylic acid, or any combination thereof.

One skilled in the relevant art would recognize that the teachings herein may be utilized with wounded or intact skin, or on mucous membranes, including but not limited to oral, bronchial, vaginal, rectal, uterine, urethral, optic, ophthalmologic, pleural, nasal, or the like.

In some embodiments, the pharmaceutically acceptable vehicle may further comprise at least a therapeutically effective amount of a first active agent and a therapeutically effective amount of a second active agent, the second active agent different than the first active agent, the first and the second active agents stored in the at least one active agent reservoir 34 of the iontophoresis delivery device 8.

In some embodiments, the first active agent is selected from an analgesic and the second active agent is selected from an antihistamine drug. In some other embodiments, the first active agent is selected from an analgesic and the second active agent is selected from a steroid. In some other embodiments, the first active agent is selected from an analgesic and the second active agent is selected from a vasoconstrictor drug. The pharmaceutically acceptable vehicle comprising the first and the second active agents may be stored in the at least one active agent reservoir. In some embodiments, the pharmaceutically acceptable vehicle may include an organic phase for storing the first active agent, and an aqueous phase for storing the second active agent.

The one or more active agents 36, 40, 42 may be mixed first with one component of the pharmaceutically acceptable vehicle (e.g., a surfactant, a nonpolar solvent, a polar agent. and the like) and then with other components of the composition. Alternatively, it may be mixed with a mixture of more than one component of the pharmaceutically acceptable vehicle (e.g., a mixture of a first surfactant and a nonpolar solvent, or a mixture of a second surfactant and a polar agent, a fluid carrier and a non-phospholipid filler component). Alternatively, it may be mixed with a particular phase of the pharmaceutically acceptable vehicle (e.g., a lipophilic phase, a hydrophilic phase, a disperse phase, a continuous phase, and the like). In certain embodiments, the one or more active agents 36, 40, 42 need be dissolved in a solvent before being mixed with the pharmaceutically acceptable vehicle.

In certain embodiments, the vehicle may include a complex of an active agent with a cyclodextrin. The complex of the active agent with the cyclodextrin may be used, for example, to enhance stability and/or iontophoretic delivery of the active agent. In certain such embodiments, a complex comprising an active agent and a cyclodextrin may be delivered iontophoretically into or through a biological interface of a subject into an underlying tissue of the subject or into a circulatory system of the subject. In certain other such embodiments, an active agent may be delivered iontophoretically from a complex of the active agent with a cyclodextrin into or through a biological interface of a subject into an underlying tissue of the subject or into a circulatory system of the subject. Examples of suitable cyclodextrins include an α-cyclodextrin, a β-cyclodextrin, a γ-cyclodextrin, and the like. In certain embodiments, a cyclodextrin may be a natural cyclodextrin. In certain other embodiments, a cyclodextrin may be a synthetic cyclodextrin. In yet other embodiments, a cyclodextrin may be a modified cyclodextrin. In certain such embodiments, the cyclodextrin may be modified to increase external surface charge of the structure. In yet other such embodiments, the cyclodextrin may be modified to increase the hydrophobic character of the interior cavity of the structure.

In certain embodiments according to methods described herein, an active agent having limited solubility at the concentration at which it is stored within an active agent reservoir of an iontophoretic device, as described, may be complexed with a cyclodextrin to improve solubility during storage and delivery. The soluble polar cyclodextrin complex of the active agent may, in certain embodiments, be iontophoretically delivered to or through a biological interface and/or into a body tissue, thereafter dissociating to release the active agent into the tissue. Certain uses of cyclodextrins to increase solubility of active agents to enhance iontophoretic delivery have been described. See, for example, U.S. Pat. No. 5,068,226, herein incorporated by reference in its entirety.

The characteristics of cyclodextrins may be useful not only to enhance aqueous solubility of poorly soluble agents, but may also serve to protect and stabilize agents from adverse effects of the environment into which they may be placed, or in which they may be stored. For example, agents that may be susceptible to degradation in an aqueous environment, or by exposure to additional components of an aqueous medium, may be prepared as cyclodextrin complexes, thus protected by storage within the apolar environment of the interior of the cyclodextrin until it is administered to a subject to be treated.

In certain embodiments, an active agent delivered by any of the iontophoretic methods described herein may be susceptible to oxidation at or by an electrode, such as a carbon electrode, during iontophoretic delivery. In such embodiments, the active agent may be stored in the iontophoretic device 8 as a complex with a cyclodextrin. Incorporation of the active agent into the interior of the toroidal structure protects the active agent from oxidative effects of the active electrode during iontophoretic delivery of the agent. In further such embodiments, entrapment of the active agent within the cavity of the cyclodextrin not only protects it from the oxidative effects that would occur upon exposure to the electrode, but may also protect the active agent from exposure to reactants that may be present in the aqueous environment. In other such embodiments, entrapment of the active agent in the cyclodextrin cavity may sterically constrain the active agent to limit chemical reactions, such as degradative reactions. Upon delivery to or through a biological interface 18 and/or into a tissue, the active agent dissociates from the complex with the cyclodextrin. In certain embodiments, the active agent may be released from the cyclodextrin complex at the interface and delivered through the interface into the underlying tissue. In certain other embodiments, the active agent-cyclodextrin complex may be delivered through the interface into the underlying tissue, where the active agent dissociates from the complex, as described elsewhere herein. Active agents that may be complexed with cyclodextrins to prevent oxidation during iontophoretic delivery, as described herein, include, without limitation, epinephrine, norepinephrine, derivatives and analogs thereof, and-caine type anesthetic agents or pain killers.

In certain embodiments, cyclodextrins may be chemically modified according to methods known in the art to increase their surface charge or polarity to enhance their utility in iontophoretic methods as disclosed herein. In certain such embodiments, for example, the external charge on the cyclodextrin may be increased to improve the efficiency of iontophoretic delivery. In certain other embodiments, wherein β-cyclodextrin is preferred over other cyclodextrins, aqueous solubility of the β-cyclodextrin may be improved. In certain such embodiments, the cavity of β-cyclodextrin may be preferred as providing optimal characteristics for the binding of particular active agents. In certain embodiments, derivatives of β-cyclodextrin may include, without limitation, hydrophilic cyclodextrins, such as 2,6-dimethyl-β-cyclodextrin and hydroxypropyl-β-cyclodextrin; hydrophobic cyclodextrins, such as 2,6-diethyl-β-cyclodextrin; and ionizable cyclodextrins, such as carboxymethyl-β-cyclodextrin, sulfated-β-cyclodextrin, and sulfobutylether-β-cyclodextrin. The use of charge-bearing cyclodextrins to increase the iontophoretic transport of active agents through the skin of a subject have been described. See, for example, U.S. Pat. No. 5,068,226, incorporated by reference herein in its entirety.

In certain further embodiments, cyclodextrins may be chemically modified according to methods known in the art to enhance the ability of the interior cavity to bind certain classes of active agents. For example, addition of alkyl groups to the interior cavity portion of natural cyclodextrins may enhance the ability of such modified cyclodextrins to increase the solubility and delivery of certain agents. In certain embodiments, for example, a cyclodextrin may be a methyl-β-cyclodextrin, a species with an enhanced ability over that of unmodified β-cyclodextrin to complex lipophilic molecules. Methods known in the art may be used to chemically modify cyclodextrins, and such modified cyclodextrins may be used to enhance the formation of complexes with a variety of active agents or chemical compounds for iontophoretic delivery according to methods disclosed herein.

In certain embodiments, cyclodextrins may be used as permeation enhancers to improved delivery of one or more active agents 36, 40, 42 through a biological interface 18.

The pharmaceutical vehicle may include a plurality of vesicles in the form of a plurality of liposomes, nisomes, ethasomes, transfersomes, virosomes, cyclic oligosaccharides, non ionic surfactant vesicles, phospholipid surfactant vesicles, micelles, microspheres, and the like, or other small lipid-based vehicles. One skilled in the relevant art would readily understand such formulations, and appreciate that they may be used by incorporation into the reservoir of the delivery device, or formulated to be applied directly to a subject's body surface. For example, liposomes may be microscopic vesicles having a lipid wall comprising a lipid bilayer, and may be preferred for poorly soluble or insoluble active agents. Liposomal formulations may be cationic, anionic, or neutral preparations. Materials and methods of making such vesicle preparations are well known in the art. See, for example, Conacher et al., “Niosomes as Immunological Adjuvants” Synthetic Surfactant Vesicles (Ed. I. F. Uchegbu) International Publishers Distributors Ltd. Singapore, pp. 185-205 (2001); Okumes et al., “Vesicle Encapsulation Studies Reveal that that Single Molecule Ribozyme Heterogeneities Are Intrinsic” Biophysical Journal, 87, pp. 2798-2806 (2004); and Sihorkar et al., “Polysaccharide Coated Niosomes for Oral Drug Delivery: Formulation and In Vitro Stability Studies” Pharmazie 55(2), pp. 107-113 (February 2000).

In certain embodiments, micelles may be used as a vehicle. As one skilled in the relevant art would appreciate, micelles are comprised of surfactant molecules arranged with polar head groups forming an outer shell, while the hydrophobic hydrocarbon chains are oriented toward the middle of the sphere, forming a core. Examples of surfactants useful for making micelles include potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10, nonoxynol 30, and the like.

In some embodiments, the iontophoretic drug delivery device 8 for providing transdermal delivery of one or more therapeutic active agents 36, 40, 42 to a biological interface 18 includes an active electrode assembly 12 including at least one active electrode element 24 and at least one active agent reservoir 34. The at least one active agent reservoir 34 includes a pharmaceutically acceptable vehicle comprising a plurality of first vesicles selected from liposomes, nisomes, ethasomes, transfersomes, virosomes, cyclic oligosaccharides, non ionic surfactant vesicles, and phospholipid surfactant vesicles. At least some of the first vesicles include one or more therapeutic active agents 36, 40, 42. The at least one active electrode element 24 is operable to provide an electromotive force to drive at least some of the pharmaceutically acceptable vehicle comprising the plurality of first vesicles, from the at least one active agent reservoir 34 to the biological interface 18. In some embodiments, the one or more therapeutic active agents 36, 40, 42 are selected from immuno-adjuvants, immuno-modulators, immuno-response agents, immuno-stimulators, specific immuno-stimulators, non-specific immuno-stimulators, and immuno-suppressants, or combinations thereof. In some other embodiments, the one or more therapeutic active agents 36, 40, 42 are selected from vaccines, agonist, antagonist, opioid agonist, opioid antagonist, antigens, adjuvants, immunological adjuvants, immunogens, tolerogens, allergens, toll-like receptor agonists, and toll-like receptor antagonists, or combinations thereof. In some further embodiments, the one or more therapeutic active agent 36, 40, 42 are selected from centbucridine, tetracaine, Novocaine® (procaine), ambucaine, amolanone, amylcaine, benoxinate, betoxycaine, carticaine, chloroprocaine, cocaethylene, cyclomethycaine, butethamine, butoxycaine, carticaine, dibucaine, dimethisoquin, dimethocaine, diperodon, dyclonine, ecogonidine, ecognine, euprocin, fenalcomine, formocaine, hexylcaine, hydroxyteteracaine, leucinocaine, levoxadrol, metabutoxycaine, methyl chloride, myrtecaine, butamben, bupivicaine, mepivacaine, beta-adrenoceptor antagonists, opioid analgesics, butanilicaine, ethyl aminobenzoate, fomocine, hydroxyprocaine, isobutyl p-aminobenzoate, naepaine, octacaine, orthocaine, oxethazaine, parenthoxycaine, phenacine, phenol, piperocaine, polidocanol, pramoxine, prilocalne, propanocaine, proparacaine, propipocaine, pseudococaine, pyrrocaine, salicyl alcohol, parethyoxycaine, piridocaine, risocaine, tolycaine, trimecaine, tetracaine, anticonvulsants, antihistamines, articaine, cocaine, procaine, amethocaine, chloroprocaine, Lidocaine® (xylocaine), marcaine, chloroprocaine, etidocaine, prilocaine, lignocaine, benzocaine, zolamine, ropivacaine, and dibucaine, or combinations thereof. In some embodiments, the pharmaceutically acceptable vehicle comprising a plurality of first vesicles is formulated as a controlled-release formulation.

In some embodiments, a substantial portion of the plurality of first vesicles includes one or more therapeutic active agents selected from amphiphilic active agents, lipophilic active agents, hydrophilic active agents, and charged hydrophilic active agents, or combinations thereof. In some embodiments, a substantial portion of the plurality of first vesicles takes the form of liposomes. In some embodiments, a substantial portion of the plurality of first vesicles takes the form of unilamella or multilamellar vesicles.

In some embodiments, a substantial portion of the plurality of first vesicles includes at least one vesicle bilayer and an encapsulated aqueous compartment. In some embodiments, a substantial portion of the plurality of first vesicles includes at least a first therapeutic active agent in the encapsulated aqueous compartment, and a second therapeutic active agent associated with the at least on vesicle bilayer. The first therapeutic active agent may be selected from one or more hydrophilic active agents and charged hydrophilic active agents, and the second therapeutic active agent may be selected from amphiphilic active agents, and lipophilic active agents. In some embodiments, at least 10% of the plurality of first vesicles includes a first therapeutic active agent. In some embodiments, at least 30% of the plurality of first vesicles includes a first therapeutic active agent. In some embodiments, at least 60% of the plurality of first vesicles includes a first therapeutic active agent.

One skilled in the relevant art would readily understand that any number of methods of making the various embodiments could be employed. For example, the compositions, including pharmaceutical compositions, may be prepared according to standard protocols, which are well known in the art. See, for example, the methods recited in Alfonso R. Gennaro ed. “Remington: The Science and Practice of Pharmacy” (19th ed. 1995). Another example includes separating the solutions into water-soluble and oil soluble components. The water-soluble components can be mixed together in one container while the oil soluble components can be mixed together in a separate container, and each mixture heated individually to form a solution. The two solutions may then be mixed and the mixture allowed to cool. Such compositions may be packaged and stored, or used directly. Other exemplary embodiments are set forth in the Examples section herein.

The vehicle (including for example a gel, organogel, and the like) and/or the therapeutic agent may be contained within a delivery device 8 such as a patch, bandage, reservoir 34, rupturable membrane, application chamber, tape, film, or other device that allows for transdermal or transmucosal delivery of an agent. In at least one embodiment, the vehicle and/or the one or more therapeutic agents 36, 40, 42 are contained within the iontophoresis device 8. In at least one embodiment, the vehicle and/or the one or more therapeutic agents 36, 40, 42 are impregnated on a substrate contained within the iontophoresis device 8. In at least one embodiment, a substrate contained within the iontophoresis device 8 is saturated with the vehicle and/or the one or more therapeutic agents 36, 40, 42. In at least one embodiment, a substrate contained within the delivery device 8 is an absorbent layer saturated with the vehicle and/or the one or more therapeutic agents 36, 40, 42. In at least one embodiment, the vehicle, and/or the one or more therapeutic agents 36, 40, 42 are contained on or in the iontophoresis device 8 that may further include an adhesive. In at least one embodiment, the vehicle and/or therapeutic agents 36, 40, 42 are combined with an adhesive for delivery to the subject. In at least one embodiment, the delivery device 8 has a medical backing 19 and pressure sensitive adhesive that allows the device 8 to adhere to a subject.

One skilled in the relevant art would recognize that multiple materials may be used for the optional absorbent layer within the iontophoresis device 8, including fabric, fibers, particulate matter, resins, polymers, or other substrate that is capable of absorbing a vehicle and/or a therapeutic agent. Some examples of materials used in constructing the absorbent layer may include, but not be limited to, cotton, polyester, polyfil, cellulose, other polymers, resins, other natural or synthetic fibers, or any combination thereof.

The iontophoretic delivery device 8 may comprise a reservoir 34, an absorbent layer, a medical backing 19, an adhesive, one or more membranes, or other structures. Such medical backing 19 and adhesive may be located on various positions of the iontophoretic delivery device 8. For example, the medical backing 19 and adhesive may be adjacent to the vehicle and/or therapeutic agent, may be opposing the vehicle and/or therapeutic agent, or may be intermixed with the vehicle and/or therapeutic agent.

In at least one embodiment, the iontophoretic delivery device 8 includes any size or shape of patch. Suitable patches include, but are not limited to, the matrix type patch, the reservoir type patch, the multi-laminate drug-in-adhesive type patch, and the monolithic drug-in-adhesive type patch. These and others are readily known in the art and are further described in Tapash, et al., “Transdermal and Topical Drug Delivery Systems” Interpharm Press, Inc (1997). For example, a matrix patch may comprise a therapeutic agent containing matrix, an adhesive backing film overlay, and a release liner. In some embodiments, one or more impermeable or semipermeable layers or membranes may be used to minimize drug migration into the backing 19. The matrix may be held against a subject's body surface by the adhesive overlay. Examples of suitable matrix materials include, but are not limited to, lipophilic polymers, hydrophilic polymers, hydrogels, or polyvinylpyrrolidone/polyethylene oxide mixtures.

In certain embodiments, the reservoir type patch may comprise a backing film coated with an adhesive, and a reservoir compartment comprising a therapeutic agent formulation, which may or may not be separated from the subject's body surface by one or more semipermeable membranes.

In certain embodiments, the monolithic drug-in-adhesive patch may comprise a drug formulation in the skin contacting adhesive layer, a backing film, and possibly a release liner. The adhesive may function to release the anesthetic and/or adhere the anesthetic matrix to the skin. The drug-in-adhesive system does not require an adhesive overlay and thus the size and height of the patch may be minimized.

In certain embodiments, the multi-laminate drug-in-adhesive patch may further incorporate one or more semipermeable membranes between two distinct drug-in-adhesive layers or multiple drug-in-adhesive layers under a single backing film.

In certain embodiments, the iontophoretic delivery device 8 may further comprises an adhesive. Adhesives are well known in the art and include, but are not limited to, polyisobutylene-based adhesives, silicone-based adhesives, and acrylic-based adhesives. The adhesive may be based on natural or synthetic rubber. In certain embodiments, the device further comprises a pressure sensitive adhesive. Pressure sensitive adhesives generally adhere to a substrate by applying light or weak force, and usually do not leave a residue when removed.

In certain embodiments, the iontophoretic delivery device 8 may be prepared by casting a fluid admixture of adhesive, therapeutic agent and vehicle onto a backing layer 19, followed by lamination of the release liner. In certain embodiments, the adhesive mixture may be cast onto the release liner, followed by lamination of the backing layer 19. In certain embodiments, the drug reservoir may be prepared in the absence of therapeutic agent and then loaded by saturating or soaking it in the therapeutic agent and/or vehicle. Other methods of making include solvent evaporation, film casting, melt extrusion, thin film lamination, die cutting, or the like.

In certain embodiments, the medical backing layer 19 may function as the primary structural element of the delivery device and may provide the device with flexibility and occlusivity (which allows the subject's body surface to become hydrated with use of the device), or permeability (which allows the subject's body surface to encounter other atmospheric agents). The backing 19 may comprise a flexible elastomeric material that protects and/or prevents the composition contained in the iontophoretic delivery device 8. In certain embodiments, the medical backing 19 and/or adhesive extends beyond the surface of the device reservoir, which allows for adherence to the subject's body even once the treatment site has become hydrated.

Additionally, in certain aspects, abrasive agents may be utilized in order to increase the transdermal or transmucosal delivery of the therapeutic agent. One skilled in the relevant art would recognize that a variety of abrasive means may be employed, such as physical, chemical, radiation, mechanical, structural or other such means. Examples of abrasive agents that may be employed include but are not limited to temperature changes; such as heat or cold; light; magnets; chemical irritants such as acids, bases, alcohols or other solvents, polymers (such as propylene glycol), salts (such as sodium laurel sulfate), plant compounds (such as from poison ivy or poison sumac), epoxy resins; vasoconstrictors such as epinephrine, adrenaline, norepinephrine; similar irritants or abrasives, and any combination thereof.

One skilled in the relevant art may also appreciate that the transdermal or transmucosal delivery device may be more or less effective depending on the location on the subject. For example, highly vascularized areas may allow for greater delivery of the therapeutic agent, as would a surface that is wounded, for example by burn, laceration or abrasion. By contrast, areas that are not highly vascularized may allow for a slower or more gradual release of the therapeutic agent.

Referring to FIGS. 2A and 2B, the active electrode assembly 12 of the iontophoretic delivery device 8 may further comprise an optional inner sealing liner (not shown) between two layers of the active electrode assembly 12, for example, between the inner ion selective membrane 30 and the inner active agent reservoir 34. The inner sealing liner, if present, would be removed prior to application of the iontophoretic device to the biological surface 18. Each of the above elements or structures will be discussed in detail below.

The active electrode element 24 is electrically coupled to a first pole 16a of the power source 16 and positioned in the active electrode assembly 12 to apply an electromotive force to transport the active agent 36, 40, 42 via various other components of the active electrode assembly 12. Under ordinary use conditions, the magnitude of the applied electromotive force is generally that required to deliver the one or more active agents according to a therapeutic effective dosage protocol. In some embodiments, the magnitude is selected such that it meets or exceeds the ordinary use operating electrochemical potential of the iontophoresis delivery device 8.

The active electrode element 24 may take a variety of forms. In one embodiment, the active electrode element 24 may advantageously take the form of a carbon-based active electrode element. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004. The carbon-based electrodes are inert electrodes in that they do not themselves undergo or participate in electrochemical reactions. Thus, an inert electrode distributes current through the oxidation or reduction of a chemical species capable of accepting or donating an electron at the potential applied to the system, (e.g., generating ions by either reduction or oxidation of water). Additional examples of inert electrodes include stainless steel, gold, platinum, capacitive carbon, or graphite.

Alternatively, an active electrode of sacrificial conductive material, such as a chemical compound or amalgam, may also be used. A sacrificial electrode does not cause electrolysis of water, but would itself be oxidized or reduced. Typically, for an anode a metal/metal salt may be employed. In such case, the metal would oxidize to metal ions, which would then be precipitated as an insoluble salt. An example of such anode includes an Ag/AgCl electrode. The reverse reaction takes place at the cathode in which the metal ion is reduced and the corresponding anion is released from the surface of the electrode.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, a membrane with pores, cavities, or interstices, particularly where the electrolyte 28 is a liquid.

In one embodiment, the electrolyte 28 comprises ionic or ionizable components in an aqueous medium, which can act to conduct current towards or away from the active electrode element. Suitable electrolytes include, for example, aqueous solutions of salts. Preferably, the electrolyte 28 includes salts of physiological ions, such as, sodium, potassium, chloride, and phosphate.

Once an electrical potential is applied, when an inert electrode element is in use, water is electrolyzed at both the active and counter electrode assemblies. In certain embodiments, such as when the active electrode assembly is an anode, water is oxidized. As a result, oxygen is removed from water while protons (H⁺) are produced. In one embodiment, the electrolyte 28 may further comprise an anti-oxidant. In some embodiments, the anti-oxidant is selected from anti-oxidants that have a lower potential than that of, for example, water. In such embodiments, the selected anti-oxidant is consumed rather than having the hydrolysis of water occur. In some further embodiments, an oxidized form of the anti-oxidant is used at the cathode and a reduced form of the anti-oxidant is used at the anode. Examples of biologically compatible anti-oxidants include, but are not limited to, ascorbic acid (vitamin C), tocopherol (vitamin E), or sodium citrate.

As noted above, the electrolyte 28 may take the form of an aqueous solution housed within a reservoir 26, or in the form of a dispersion in a hydrogel or hydrophilic polymer capable of retaining substantial amount of water. For instance, a suitable electrolyte may take the form of a solution of 0.5 M disodium fumarate: 0.5 M polyacrylic acid: 0.15 M anti-oxidant.

The inner ion selective membrane 30 is generally positioned to separate the electrolyte 28 and the inner active agent reservoir 34, if such a membrane is included within the device. The inner ion selective membrane 30 may take the form of a charge selective membrane. For example, when the active agent 36, 40, 42 comprises a cationic active agent, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na⁺) ions from the electrolyte 28, thereby increasing the transfer rate and/or biological compatibility of the iontophoresis device 8.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36. For example, the inner active agent reservoir 34 may take the form of a pouch or other receptacle, a membrane with pores, cavities, or interstices, particularly where the active agent 36 is a liquid. The inner active agent reservoir 34 further may comprise a gel matrix.

Optionally, an outermost ion selective membrane 38 is positioned generally opposed across the active electrode assembly 12 from the active electrode element 24. The outermost membrane 38 may, as in the embodiment illustrated in FIGS. 2A, 2B and 2C, take the form of an ion exchange membrane having pores 48 (only one called out in FIGS. 2A, 2B and 2C for sake of clarity of illustration) of the ion selective membrane 38 including ion exchange material or groups 50 (only three called out in FIGS. 2A, 2B and 2C for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., lidocaine), the outermost ion selective membrane 38 may take the form of a cation exchange membrane, thus allowing the passage of the cationic active agent while blocking the back flux of the anions present in the biological interface, such as skin.

The outermost ion selective membrane 38 may optionally cache active agent 40. Without being limited by theory, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of semi-permeable or microporous membrane which is selective by size. In some embodiments, such a semi-permeable membrane may advantageously cache active agent 40, for example by employing the removably releasable outer release liner to retain the active agent 40 until the outer release liner is removed prior to use.

The outermost ion selective membrane 38 may be optionally preloaded with the additional active agent 40, such as ionized or ionizable drugs or therapeutic agents and/or polarized or polarizable drugs or therapeutic agents. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness or coverage as to constitute a layer in a conventional sense of such term.

The active agent 42 may be deposited in a variety of highly concentrated forms such as, for example, solid form, nearly saturated solution form, or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

The outer release liner may generally be positioned overlying or covering further active agent 42 carried by the outer surface 44 of the outermost ion selective membrane 38. The outer release liner may protect the further active agent 42 and/or outermost ion selective membrane 38 during storage, prior to application of an electromotive force or current. The outer release liner may be a selectively releasable liner made of waterproof material, such as release liners commonly associated with pressure sensitive adhesives.

An interface-coupling medium (not shown) may be employed between the electrode assembly and the biological interface 18. The interface-coupling medium may take, for example, the form of an adhesive and/or gel. The gel may take, for example, the form of a hydrating gel. Selection of suitable bioadhesive gels is within the knowledge of one skilled in the relevant art.

In the embodiment illustrated in FIGS. 2A and 2B, the counter electrode assembly 14 comprises, from an interior 64 to an exterior 66 of the counter electrode assembly 14: a counter electrode element 68, an electrolyte reservoir 70 storing an electrolyte 72, an inner ion selective membrane 74, an optional buffer reservoir 76 storing buffer material 78, an optional outermost ion selective membrane 80, and an optional outer release liner (not shown).

The counter electrode element 68 is electrically coupled to a second pole 16b of the power source 16, the second pole 16b having an opposite polarity to the first pole 16a. In one embodiment, the counter electrode element 68 is an inert electrode. For example, the counter electrode element 68 may take the form of the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities, or interstices, particularly where the electrolyte 72 is a liquid.

The electrolyte 72 is generally positioned between the counter electrode element 68 and the outermost ion selective membrane 80, proximate the counter electrode element 68. As described above, the electrolyte 72 may provide ions or donate charges to prevent or inhibit the formation of gas bubbles (e.g., hydrogen or oxygen, depending on the polarity of the electrode) on the counter electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate, the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxy (OH⁻) or chloride (Cl⁻) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane, or a gel.

The buffer material 78 may supply ions for transfer through the outermost ion selective membrane 42 to the biological interface 18. Consequently, the buffer material 78 may, for example, comprise a salt (e.g., NaCl).

The outermost ion selective membrane 80 of the counter electrode assembly 14 may take a variety of forms. For example, the outermost ion selective membrane 80 may take the form of a charge selective ion exchange membrane. Typically, the outermost ion selective membrane 80 of the counter electrode assembly 14 is selective to ions with a charge or polarity opposite to that of the outermost ion selective membrane 38 of the active electrode assembly 12. The outermost ion selective membrane 80 is therefore an anion exchange membrane, which substantially passes anions and blocks cations, thereby prevents the back flux of the cations from the biological interface. Examples of suitable ion exchange membranes are discussed above.

Alternatively, the outermost ion selective membrane 80 may take the form of a semi-permeable membrane that substantially passes and/or blocks ions based on size or molecular weight of the ion.

The outer release liner (not shown) may generally be positioned overlying or covering an outer surface 84 of the outermost ion selective membrane 80. The outer release liner may protect the outermost ion selective membrane 80 during storage, prior to application of an electromotive force or current. The outer release liner may be a selectively releasable liner made of waterproof material, such as release liners commonly associated with pressure sensitive adhesives. In some embodiments, the outer release liner may be coextensive with the outer release liner (not shown) of the active electrode assembly 12.

The iontophoresis device 8 may further comprise an inert molding material 86 adjacent exposed sides of the various other structures forming the active and counter electrode assemblies 12, 14. The molding material 86 may advantageously provide environmental protection to the various structures of the active and counter electrode assemblies 12, 14. Enveloping the active and counter electrode assemblies 12, 14 is a housing material 90.

As best seen in FIG. 2B, the active and counter electrode assemblies 12, 14 are positioned on the biological interface 18. Positioning on the biological interface may close the circuit, allowing electromotive force to be applied and/or current to flow from one pole 16a of the power source 16 to the other pole 16b, via the active electrode assembly, biological interface 18 and counter electrode assembly 14.

In use, the outermost active electrode ion selective membrane 38 may be placed directly in contact with the biological interface 18. Alternatively, an interface-coupling medium (not shown) may be employed between the outermost active electrode ion selective membrane 22 and the biological interface 18. The interface-coupling medium may take, for example, the form of an adhesive and/or gel. The gel may take, for example, the form of a hydrating gel or a hydrogel. If used, the interface-coupling medium should be permeable by the active agent 36, 40, 42.

In some embodiments, the power source 16 is selected to provide sufficient voltage, current, and/or duration to ensure delivery of the one or more active agents 36, 40, 42 from the reservoir 34 and across a biological interface (e.g., a membrane) to impart the desired physiological effect. The power source 16 may take the form of one or more chemical battery cells, super- or ultra-capacitors, fuel cells, secondary cells, thin film secondary cells, button cells, lithium ion cells, zinc air cells, nickel metal hydride cells, and the like. The power source 16 may, for example, provide a voltage of 12.8 V DC, with tolerance of 0.8 V DC, and a current of 0.3 mA. The power source 16 may be selectively electrically coupled to the active and counter electrode assemblies 12, 14 via a control circuit, for example, via carbon fiber ribbons. The iontophoresis device 8 may include discrete and/or integrated circuit elements to control the voltage, current, and/or power delivered to the electrode assemblies 12, 14. For example, the iontophoresis device 8 may include a diode to provide a constant current to the electrode elements 24, 68.

As suggested above, the one or more active agents 36, 40, 42 may take the form of one or more ionic, cationic, ionizeable, and/or neutral drugs or other therapeutic agents. Consequently, the poles or terminals of the power source 16 and the selectivity of the outermost ion selective membranes 38, 80 and inner ion selective membranes 30, 74 are selected accordingly.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small more highly charged active agents under the influence of these forces. These higher molecular weight active agents may thus be carried through the biological interface into the underlying tissues primarily via electroosmotic solvent flow. In certain embodiments, the high molecular weight polyelectrolytic active agents may be proteins, polypeptides, or nucleic acids. In other embodiments, the active agent may be mixed with another agent to form a complex capable of being transported across the biological interface via one of the motive methods described above.

In some embodiments, the transdermal drug delivery system 6 includes an iontophoretic drug delivery device 8 for providing transdermal delivery of one or more therapeutic active agents 36, 40, 42 to a biological interface 18. The delivery device 8 includes active electrode assembly 12 including at least one active agent reservoir and at least one active electrode element operable to provide an electromotive force to drive an active agent from the at least one active agent reservoir. The delivery device 8 may include a counter electrode assembly 14 including at least one counter electrode element 68, and a power source 16 electrically coupled to the at least one active and the at least one counter electrode elements 20, 68. In some embodiments, the iontophoretic drug delivery 8 may further include one or more active agents 36, 40, 42 loaded in the at least one active agent reservoir 34.

As shown in FIG. 2C, the delivery device 8 may further include a substrate 10 including a plurality of microneedles 17 in fluidic communication with the active electrode assembly 12, and positioned between the active electrode assembly 12 and the biological interface 18. The substrate 10 may be positioned between the active electrode assembly 12 and the biological interface 18. In some embodiments, the at least one active electrode element 20 is operable to provide an electromotive force to drive an active agent 36, 40, 42 from the at least one active agent reservoir 34, through the plurality of microneedles 17, and to the biological interface 18.

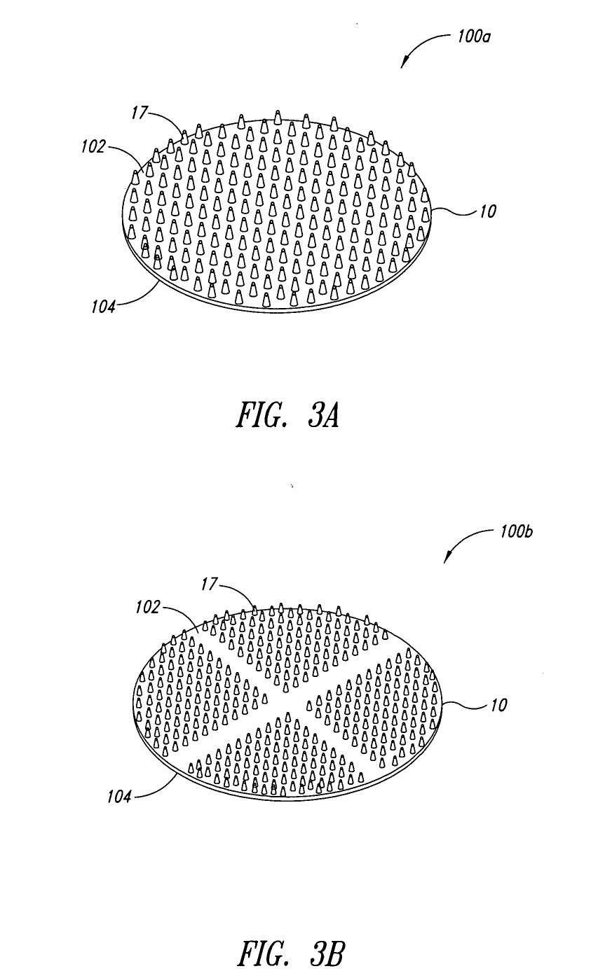

As shown in FIGS. 3A and 3B, the substrate 10 includes a first side 102 and a second side 104 opposing the first side 102. The first side 102 of the substrate 10 includes a plurality of microneedles 17 projecting outwardly from the first side 102. The microneedles 17 may be individually provided or formed as part of one or more arrays. In some embodiments, the microneedles 17 are integrally formed from the substrate 10. The microneedles 17 may take a solid and permeable form, a solid and semi-permeable form, and/or a solid and non-permeable form. In some other embodiments, solid, non-permeable, microneedles may further comprise grooves along their outer surfaces for aiding the transdermal delivery of one or more active agents. In some other embodiments, the microneedles 17 may take the form of hollow microneedles. In some embodiments, the hollow microneedles may be filled with ion exchange material, ion selective materials, permeable materials, semi-permeable materials, solid materials, and the like.

The microneedles 17 are used, for example, to deliver a variety of pharmaceutical compositions, molecules, compounds, active agents, and the like to a living body via a biological interface, such as skin or mucous membrane. In certain embodiments, pharmaceutical compositions, molecules, compounds, active agents, and the like may be delivered into or through the biological interface. For example, in delivering pharmaceutical compositions, molecules, compounds, active agents, and the like via the skin, the length of the microneedle 17, either individually or in arrays 100a, 100b, and/or the depth of insertion may be used to control whether administration of a pharmaceutical compositions, molecules, compounds, active agents, and the like is only into the epidermis, through the epidermis to the dermis, or subcutaneous. In certain embodiments, the microneedle 17 may be useful for delivering high-molecular weight active agents, such as those comprising proteins, peptides and/or nucleic acids, and corresponding compositions thereof. In certain embodiments, for example wherein the fluid is an ionic solution, the microneedles 17 can provide electrical continuity between the power source 16 and the tips of the microneedles 17. In some embodiments, the microneedles 17, either individually or in arrays 100a, 100b, may be used to dispense, deliver, and/or sample fluids through hollow apertures, through the solid permeable or semi permeable materials, or via external grooves. The microneedles 17 may further be used to dispense, deliver, and/or sample pharmaceutical compositions, molecules, compounds, active agents, and the like by iontophoretic methods, as disclosed herein.

Accordingly, in certain embodiments, for example, a plurality of microneedles 17 in an array 100a, 100b may advantageously be formed on an outermost biological interface-contacting surface of a transdermal drug delivery system 6. In some embodiments, the pharmaceutical compositions, molecules, compounds, active agents, and the like delivered or sampled by such a system 6 may comprise, for example, high-molecular weight active agents, such as proteins, peptides, and/or nucleic acids.

In some embodiments, a plurality of microneedles 17 may take the form of a microneedle array 100a, 100b. The microneedle array 100a, 100b may be arranged in a variety of configurations and patterns including, for example, a rectangle, a square, a circle (as shown in FIG. 3A), a triangle, a polygon, a regular or irregular shapes, and the like. The microneedles 17 and the microneedle arrays 100a, 100b may be manufactured from a variety of materials, including ceramics, elastomers, epoxy photoresist, glass, glass polymers, glass/polymer materials, metals (e.g., chromium, cobalt, gold, molybdenum, nickel, stainless steel, titanium, tungsten steel, and the like), molded plastics, polymers, biodegradable polymers, non-biodegradable polymers, organic polymers, inorganic polymers, silicon, silicon dioxide, polysilicon, silicon rubbers, silicon-based organic polymers, superconducting materials (e.g., superconductor wafers, and the like), and the like, as well as combinations, composites, and/or alloys thereof. Techniques for fabricating the microneedles 17 are well known in the art and include, for example, electro-deposition, electro-deposition onto laser-drilled polymer molds, laser cutting and electro-polishing, laser micromachining, surface micro-machining, soft lithography, x-ray lithography, LIGA techniques (e.g., X-ray lithography, electroplating, and molding), injection molding, conventional silicon-based fabrication methods (e.g., inductively coupled plasma etching, wet etching, isotropic and anisotropic etching, isotropic silicon etching, anisotropic silicon etching, anisotropic GaAs etching, deep reactive ion etching, silicon isotropic etching, silicon bulk micromachining, and the like), complementary-symmetry/metal-oxide semiconductor (CMOS) technology, deep x-ray exposure techniques, and the like. See for example, U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; and 6,939,311. Some or all of the teachings therein may be applied to microneedle devices, their manufacture, and their use in iontophoretic applications. In some techniques, the physical characteristics of the microneedles 17 depend on, for example, the anodization conditions (e.g., current density, etching time, HF concentration, temperature, bias settings, and the like) as well as substrate properties (e.g., doping density, doping orientation, and the like).

The microneedles 17 may be sized and shaped to penetrate the outer layers of skin to increase its permeability and transdermal transport of pharmaceutical compositions, molecules, compounds, active agents, and the like. In some embodiments, the microneedles 17 are sized and shaped with an appropriate geometry and sufficient strength to insert into a biological interface (e.g., the skin or mucous membrane on a subject, and the like), and thereby increase a trans-interface (e.g., transdermal) transport of pharmaceutical compositions, molecules, compounds, active agents, and the like.

In some embodiments, the present disclosure further includes kits, including one or more compositions comprising a vehicle and a therapeutic agent that may be packaged together or separately, and that may be pre-mixed or mixed by the user, or used separately. The kits may further comprise a iontophoretic delivery device 8, including a patch, bandage, film, or other device. The kits generally include instructions for how to use the device and/or vehicle and agent compositions. Such instructions may be printed on the package, or be present as a package insert. In certain embodiments, the instructions are present on an electronic storage data file present on a suitable computer readable and/or writable storage medium (for example, a CD-ROM).

In some embodiments, the present disclosure is also directed to an article of manufacture for transdermal administration of medication by iontophoresis. The article of manufacture includes an iontophoretic drug delivery device 8, at least one dosage form, and a package insert.

The iontophoretic drug delivery device 8 includes an active electrode assembly 12 including at least one active electrode element 24 and at least one active agent reservoir 34. The at least one active agent reservoir 34 includes a pharmaceutically acceptable vehicle including at least one surfactant, at least one nonpolar solvent, and at least one polar agent. In some embodiments, the at least one active electrode element 24 is operable to provide an electromotive force to drive one or more active agents from the at least one active agent reservoir 34. In some embodiments, the at least one dosage form includes one or more active agents selected from analgesics, anesthetics, or combinations thereof. The at least one dosage form may be loaded in the pharmaceutically acceptable vehicle.

The package insert provides instructions for transdermally administering, to a subject in need of pain therapy, a therapeutically effective amount of the at least one dosage form.

lontophoresis generally uses a direct current of either positive or negative polarity to transfer drugs or transport a pharmaceutical vehicle of the corresponding polarity into, for example, the biological interface 18 of a subject. The amount of current applied over a period of time determines the amount of drug and/or vehicle delivered and is usually expressed as milliampere per minute (mA-minutes). For example, applying a current (I) of 4 mA for a time (T) of 10 minutes corresponds to a 40 mAmin dose. Using Faraday's law, the amount of drug deliver (D) can be determine by the relationship D=(IT)/ZF, where I is the current, T is the time, Z is the valance of the drug and F is Faradays constant. For example, applying a current of 4 mA for 10 minutes to a drug having a valance of (⁺1) corresponds to a theoretical delivery rate of about 3×10³ nmol·min⁻¹. Applying a current of 1 mA for 10 minutes to a drug having a valance of (⁺1) corresponds to a theoretical delivery rate of about 6×10² nmol·min¹. In some embodiments, the package insert further includes a table of current dose settings in mA-minutes for delivering a therapeutically effective amount of the at least one dosage form.

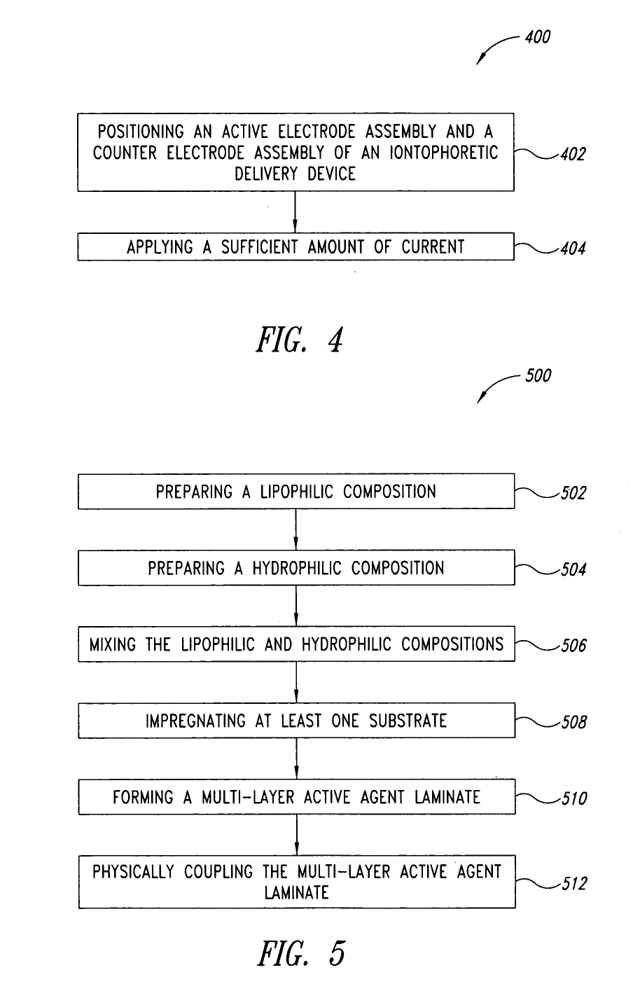

FIG. 4 shows an exemplary method 400 for transdermal administration of at least one analgesic or anesthetic by iontophoresis.

At 402, the method includes positioning an active electrode assembly and a counter electrode assembly of an iontophoretic delivery device on a biological interface of a subject. In some embodiments, the active electrode assembly includes an active agent reservoir 34 comprising at least one analgesic or anesthetic active agent 36, 40, 42 carried by a pharmaceutically acceptable vehicle. In some embodiments, the pharmaceutically acceptable vehicle comprises at least one surfactant, at least one nonpolar solvent, and at least one polar agent.

At 404, the method includes applying a sufficient amount of current to transport the at least one analgesic or anesthetic active agent 36, 40, 42 from the active agent reservoir 34, to the biological interface 18 of the subject, and to administer a therapeutically effective amount of the at least one analgesic or anesthetic active agent 36, 40, 42 to produce analgesic or anesthetic therapy in the subject for a limited period of time. In some embodiments, the at least one analgesic or anesthetic active agent 36, 40, 42 is selected from alfentanil, codeine, COX-2 inhibitors, opiates, opioid agonist, opioid antagonist, diamorphine, fentanyl, meperidine, methadone, morphine morphinomimetics, naloxone, nonsteroidal anti-inflammatory drugs (NSAIDs), oxycodone, remifentanil, sufentanil, and tricyclic antidepressants, or combinations thereof. In some other embodiments, the at least one analgesic or anesthetic active agent 36, 40, 42, further includes one or more active agents selected from immuno-adjuvants, immuno-modulators, immuno-response agents, immuno-stimulators, specific immuno-stimulators, non-specific immuno-stimulators, and immuno-suppressants vaccines, agonist, antagonist, opioid agonist, opioid antagonist, antigens, adjuvants, immunological adjuvants, immunogens, tolerogens, allergens, toll-like receptor agonists, and toll-like receptor antagonists, or combinations thereof.

In some embodiments, applying a sufficient amount of current to transport the at least one analgesic or anesthetic active agent 36, 40, 42 includes providing sufficient voltage and current to deliver a therapeutically effective amount of the at least one analgesic or anesthetic active agent 36, 40, 42 carried by the pharmaceutically acceptable vehicle comprising the at least one surfactant, the at least one nonpolar solvent, and the at least one polar agent; from the active agent reservoir 34 to the biological interface 18 of the subject.

In some other embodiments, applying a sufficient amount of current to transport the at least one analgesic or anesthetic active agent 36, 40, 42 includes providing a sufficient voltage and current to the active electrode assembly 12 to substantially achieve sustained-delivery or controlled-delivery of a therapeutically effective amount of the at least one analgesic or anesthetic active agent 36, 40, 42 carried by the pharmaceutically acceptable vehicle comprising the at least one surfactant, the at least one nonpolar solvent, and the at least one polar agent; from the active agent reservoir 34 to the biological interface 18 of the subject.

FIG. 5 shows an exemplary method 500 of making an active agent laminate for an iontophoretic drug delivery device 8 that provides transdermal delivery of one or more therapeutic active agents 36, 40, 42 to a biological interface 18.

At 502, the method includes preparing a lipophilic composition comprising a first surfactant and a nonpolar solvent. The lipophilic composition may further comprise one or more lipophilic active agents. In some embodiments, preparing a lipophilic composition further includes combining the first surfactant, the nonpolar solvent, and the one or more lipophilic active agents.

At 504, the method includes preparing a hydrophilic composition comprising a second surfactant and a polar agent. The hydrophilic composition may further comprise one or more hydrophilic active agents. In some embodiments, preparing a hydrophilic composition further includes combining the second surfactant, the nonpolar agent, and the one or more hydrophilic active agents.

At 506, the method includes mixing the lipophilic composition and the hydrophilic composition using a high-shear mixer to form a pharmaceutically acceptable vehicle having a lipophilic phase and a hydrophilic phase. Examples of high-shear mixers include high-shear blenders, two syringes connected by a Luer lock, an electronic pestle and mortar, and the like. High-shear mixing helps to homogenize the mixture, and form the organogels. For small volumes, high shear mixing may require passing the lipophilic phase and hydrophilic phase mixture through an interconnected Luer-Lok to Luer-Lok syringe combination several times.

A pluronic lecithin organogel can be prepared by, for example, combining a 50:50 lecithin:isopropylpalmitate solution with a mixuture of Pluronic F 127. See, for example, “International Journal of Pharmaceutical Compounding” 8:59 (January /Februray 2004). The 50:50 (lecithin:isopropylpalmitate) solution can be prepared by mixing approximately 0.2 g sorbic acid, 50 g of soy lecithin and 50 g of isopropyl palmitate. The Pluronic F127 can be prepared by mixing approximately 0.2 g sorbic acid, 20 g of Pluronic F127 and sufficient purified water to make 100 mL. The lecithin/isopropyl palmitate solution and the Pluronic F127 can then be combined using a high-shear mixer to form a pluronic lecithin organogel.

A lecithin organogel could also be made, for example, by dissolving lecithin in n-decane, or other organic solvent. A therapeutic agent or agents 36, 40, 42, such as a—caine drug would then be added, possibly along with epinephrine to saturation. The solution would then be stirred to ensure complete mixing. Finally, a small amount of water would be added to induce formation of the lecithin organogel. Such organogel could be added to a passive system for delivery across skin or mucous membranes of a subject.

If such therapeutic organogel were to be packaged in a patch or other delivery device, the medical backing 19 would be cut and the adhesive exposed by peeling the release liner on the inside. The absorbent pad would be stuck to the backing 19. The formulated gel would then be dispersed onto the patch and appropriate sealing techniques would be used to create the simple, stabile unit.

For application to a subject, the packaging would be removed, the skin possibly would be abraded by using a rough material to allow better permeation of the gel, and the patch would be placed on the site and left until significant anesthesia or other effect was achieved.

At 508, the method includes impregnating at least one substrate with the pharmaceutically acceptable vehicle. In at least one embodiment, a substrate contained within the iontophoresis device 8 is impregnated with the vehicle and/or the one or more therapeutic agents 36, 40, 42.

At 510, the method includes forming a multi-layer active agent laminate including the at least one substrate with the pharmaceutically acceptable vehicle and at least one delivery rate controlling membrane. In some embodiments, forming at least one multi-layer active agent laminate includes physically coupling the at least one delivery rate controlling membrane to the at least one active agent reservoir 34 wherein the delivery rate controlling membrane controls the rate of delivery of the pharmaceutically acceptable vehicle.

At 512, the method includes physically coupling the multi-layer active agent laminate to an active electrode assembly of an iontophoretic drug delivery device, the active electrode assembly 12 including at least one active electrode element 24 operable to provide an electromotive force to drive at least some of the pharmaceutically acceptable vehicle from the multi-layer active agent laminate to the biological interface 18.

The above description of illustrated embodiments, including what is described in the Abstract, is not intended to be exhaustive or to limit the claims to the precise forms disclosed. Although specific embodiments and examples are described herein for illustrative purposes, various equivalent modifications can be made without departing from the spirit and scope of the disclosure, as will be recognized by those skilled in the relevant art. The teachings provided herein can be applied to other agent delivery systems and devices, not necessarily the exemplary iontophoresis active agent system and devices generally described above. For instance, some embodiments may include additional structure. For example, some embodiments may include a control circuit or subsystem to control a voltage, current, or power applied to the active and counter electrode elements 20, 68. Also for example, some embodiments may include an interface layer interposed between the outermost active electrode ion selective membrane 22 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir having a drug solution that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the voltage source; a second electrolyte holding part that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the voltage source; a first electrolyte reservoir having an electrolyte that is in contact with the first electrode member and to which is applied a voltage via the first electrode member; a first anion-exchange membrane that is placed on the forward surface of the first electrolyte holding part; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the voltage source; a second electrolyte holding part having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte holding part and the cation-exchange membrane; a second anion-exchange membrane placed against the forward surface of the third electrolyte reservoir; and a second cover or container that accommodates these members.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No WO03037425; U.S. patent application Ser. No. 10/488,970, filed Mar. 9, 2004; Japanese patent application 2004/317317, filed Oct. 29, 2004; U.S. provisional patent application Ser. No. 60/627,952, filed Nov. 16, 2004; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; Japanese patent application Serial No. 2005-081220, filed Mar. 22, 2005; U.S. Provisional Patent Application No. 60/722,136 filed Sep. 30, 2005; U.S. Provisional Patent Application No. 60/754,688 filed Dec. 29, 2005; U.S. Provisional Patent Application No. 60/755,199 filed Dec. 30, 2005; and U.S. Provisional Patent Application No. 60/755,401 filed Dec. 30, 2005.

As one skill in the relevant art would readily appreciate, the present disclosure comprises methods of treating a subject by any of the compositions and/or methods described herein.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs, or other structures. Still other embodiments may employ additional ones of the membranes, reservoirs, and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

What is claimed is:

1. An iontophoretic drug delivery device for providing transdermal delivery of one or more therapeutic active agents to a biological interface, comprising:
an active electrode assembly including at least one active electrode element; and
at least one active agent reservoir, the at least one active agent reservoir including a pharmaceutically acceptable vehicle for transporting one or more active agents, the pharmaceutically acceptable vehicle comprising
at least one surfactant,
at least one nonpolar solvent, and
at least one polar agent;
the at least one active electrode element operable to provide an electromotive force for driving the pharmaceutically acceptable vehicle from the at least one active agent reservoir to the biological interface.

2. The iontophoretic drug delivery device of claim 1 wherein the at least one surfactant comprises at least a glycerol residue moiety, one or more C₁₀-C₂₀ hydrocarbon chain moieties, and a moiety selected from phosphatidic acids, phosphatidylcholines, lyso-phosphatidylcholines, phosphatidylethanol amines, lysol-phosphatidylethanol amines, phosphatidylserines, and phosphatidylinositols.

3. The iontophoretic drug delivery device of claim 1 wherein the at least one surfactant is selected from one or more emulsifying agents, amphoteric surfactants, non-ionic surfactants, ionic surfactants, acetone-insoluble phosphatides, phospholipids, sorbitan esters, sorbitan monoester, and polyoxypropylene-polyoxethylene block copolymers, or combinations thereof.

4. The iontophoretic drug delivery device of claim 1 wherein the at least one surfactant is selected from one or more amphiphiles, biocompatible surfactants, ether lipids, fluoro-lipids, polyhydroxyl lipids, polymerized liposomes, lecithin, hydrogenated lecithin, naturally occurring lecithin, egg lecithin, hydrogenated egg lecithin, soy lecithin, hydrogenated soy lecithin, vegetable lecithin, sorbitan esters, sorbirant monoesters, sorbitan monolaurate, sorbitan monooleate, sorbitan monopalmitate, sorbitan monostearate, sorbitan monostearate-palmitate, sorbitan sexquioleate, sorbitan tristearate, sorbitan trioleate, diacylglycerols, gangliosides, glycerophospholipids, lysophospholipids, mixed-chain phospholipids, pegylated phospholipids, phosphatidic acids, phosphatidylcholines, phosphatidylethanolamines, phosphatidylinositols, phosphocholines, phosphoethanolamines, phosphoglycerols, phosphoserines, phytosphingosines, poloxamers, polyoxypropylene-polyoxethylene block copolymers, and sphingosines, or combinations thereof.

5. The iontophoretic drug delivery device of claim 1, wherein the at least one surfactant of the pharmaceutically acceptable vehicle comprises:
at least a first surfactant and a second surfactant; the first surfactant selected from phosphatidylcholines, and the second surfactants selected from poloxamers and polyoxypropylene-polyoxethylene block copolymers.

6. The iontophoretic drug delivery device of claim 1 wherein the at least one nonpolar solvent is selected from: organic solvents; vegetable oils; saturated or unsaturated, linear or branched, substituted or unsubstituted alkanes; ethers; esters; fatty acids; and amines.

7. The iontophoretic drug delivery device of claim 1 wherein the at least one nonpolar solvent is selected from ethyl laureate, ethyl myristate, isopropyl myristate, isopropyl palmitate, cyclopentane, cyclooctane, trans-decalin, trans-pinane, n-pentane, n-hexane, n-hexadecane, and tripropylamine.

8. The iontophoretic drug delivery device of claim 1 wherein the at least one polar agent is selected from water, alcohols, polyalcohols, glycerols, polyglycerols, glycols, polyglycols, ethylene glycol, and formamide.

9. The iontophoretic drug delivery device of claim 1 wherein the pharmaceutically acceptable vehicle takes the form of a colloidal dispersion having an aqueous phase and a lipid phase.

10. The iontophoretic drug delivery device of claim 1 wherein the pharmaceutically acceptable vehicle takes the form of a gel.

11. The iontophoretic drug delivery device of claim 1 wherein the pharmaceutically acceptable vehicle takes the form of an organogel.

12. The iontophoretic drug delivery device of claim 1 wherein the pharmaceutically acceptable vehicle has an organic phase and an aqueous phase.

13. The iontophoretic drug delivery device of claim 1 wherein the pharmaceutically acceptable vehicle has a dispersed phase and a continuous phase.

14. The iontophoretic drug delivery device of claim 1, wherein the at least one surfactant of the pharmaceutically acceptable vehicle comprises:
at least a first surfactant and a second surfactant; wherein the first surfactant is selected from lecithin, hydrogenated lecithin, naturally occurring lecithin, egg lecithin, hydrogenated egg lecithin, soy lecithin, hydrogenated soy lecithin, and vegetable lecithin; the second surfactant is selected from poloxamers and polyoxypropylene-polyoxethylene block copolymers;
the at least one nonpolar solvent is selected from ethyl laureate, ethyl myristate, isopropyl myristate, isopropyl palmitate, cyclopentane, cyclooctane, trans-decalin, trans-pinane, n-pentane, n-hexane, n-hexadecane, and tripropylamine; and
the at least one polar agent is selected from water, alcohols, polyalcohols, glycerol, glycerols, polyglycerols, ethylene glycol, polyglycols, and formamide.

15. The iontophoretic drug delivery device of claim 14 wherein the pharmaceutically acceptable vehicle takes the form of a lecithin organogel.

16. The iontophoretic drug delivery device of claim 14 wherein the pharmaceutically acceptable vehicle takes the form of a pluronic lecithin organogel.

17. The iontophoretic drug delivery device of claim 1 wherein the pharmaceutically acceptable vehicle is formulated as a controlled-release formulation.

18. The iontophoretic drug delivery device of claim 1, further comprising:
a therapeutically effective amount of one or more active agents stored in the at least one active agent reservoir.

19. The iontophoretic drug delivery device of claim 18 wherein the one or more active agents are selected from immuno-adjuvants, immuno-modulators, immuno-response agents, immuno-stimulators, specific immuno-stimulators, non-specific immuno-stimulators, and immuno-suppressants, or combinations thereof.

20. The iontophoretic drug delivery device of claim 18 wherein the one or more active agents are selected from vaccines, agonists, antagonist, opioid agonist, opioid antagonist, antigens, adjuvants, immunological adjuvants, immunogens, tolerogens, allergens, toll-like receptor agonists, and toll-like receptor antagonists, or combinations thereof.

21. The iontophoretic drug delivery device of claim 18 wherein the one or more active agents are selected from analgesics, anesthetics, or combinations thereof.

22. The iontophoretic drug delivery device of claim 18, further comprising:
at least a therapeutically effective amount of a first active agent and a therapeutically effective amount of a second active agent, the second active agent different than the first active agent, the first and the second active agents stored in the at least one active agent reservoir.

23. The iontophoretic drug delivery device of claim 22 wherein the first active agent is selected from an analgesic and the second active agent is selected from an antihistamine drug.

24. The iontophoretic drug delivery device of claim 22 wherein the first active agent is selected from an analgesic and the second active agent is selected from a steroid.

25. The iontophoretic drug delivery device of claim 22 wherein the first active agent is selected from an analgesic and the second active agent is selected from a vasoconstrictor drug.

26. The iontophoretic drug delivery device of claim 1, further comprising:
at least a first active agent and a second active, the second active agent different than the first active agent, the first and the second active agents stored in the at least one active agent reservoir;
wherein the pharmaceutically acceptable vehicle includes an organic phase for storing the first active agent, and an aqueous phase for storing the second active agent.

27. The iontophoretic drug delivery device of claim 1, wherein the pharmaceutically acceptable vehicle further comprises:
a complex of a cyclodextrin with at least one active agent.

28. A method of making an active agent laminate for an iontophoretic drug delivery device that provides transdermal delivery of one or more therapeutic active agents to a biological interface, comprising:
preparing a lipophilic composition comprising a first surfactant and a nonpolar solvent;
preparing a hydrophilic composition comprising a second surfactant and a polar agent;
mixing the lipophilic composition and the hydrophilic composition using a high-shear mixer to form a pharmaceutically acceptable vehicle having a lipophilic phase and a hydrophilic phase;
impregnating at least one substrate with the pharmaceutically acceptable vehicle;
forming a multi-layer active agent laminate including the at least one substrate with the pharmaceutically acceptable vehicle and at least one delivery rate controlling membrane; and
physically coupling the multi-layer active agent laminate to an active electrode assembly of an iontophoretic drug delivery device, the active electrode assembly including at least one active electrode element operable to provide an electromotive force to drive at least some of the pharmaceutically acceptable vehicle from the multi-layer active agent laminate to a biological interface.

29. The method of claim 28, wherein the lipophilic composition further comprises one or more lipophilic active agents.

30. The method of claim 29, wherein preparing a lipophilic composition further comprises:
combining the first surfactant, the nonpolar solvent, and the one or more lipophilic active agents.

31. The method of claim 28, wherein the hydrophilic composition further comprises one or more hydrophilic active agents.

32. The method of claim 31, wherein preparing a hydrophilic composition further comprises:
combining the second surfactant, the nonpolar agent, and the one or more hydrophilic active agents.

33. The method of claim 28, wherein forming at least one multi-layer active agent laminate comprises:
physically coupling the at least one delivery rate controlling membrane to the at least one active agent reservoir wherein the delivery rate controlling membrane controls the rate of delivery of the pharmaceutically acceptable vehicle.

34. An article of manufacture for transdermal administration of medication by iontophoresis, comprising:
an iontophoretic drug delivery device comprising an active electrode assembly comprising at least one active electrode element and at least one active agent reservoir, the at least one active agent reservoir including a pharmaceutically acceptable vehicle comprising at least one surfactant, at least one nonpolar solvent, and at least one polar agent, the at least one active electrode element operable to provide an electromotive force to drive one or more active agents from the at least one active agent reservoir;
at least one dosage form comprising one or more active agents selected from analgesics, anesthetics, or combinations thereof, the at least one dosage form loaded in the pharmaceutically acceptable vehicle; and
a package insert providing instructions for transdermally administering, to a subject in need of pain therapy, a therapeutically effective amount of the at least one dosage form.

35. The article of manufacture of claim 34, wherein the package insert further comprises:
a table of current dose settings in mA-minutes for delivering a therapeutically effective amount of the at least one dosage form.

36. A method for transdermal administration of at least one analgesic or anesthetic by iontophoresis, comprising:
positioning an active electrode assembly and a counter electrode assembly of an iontophoretic delivery device on a biological interface of a subject, the active electrode including an active agent reservoir comprising at least one analgesic or anesthetic active agent carried by a pharmaceutically acceptable vehicle comprising at least one surfactant, at least one nonpolar solvent, and at least one polar agent; and
applying a sufficient amount of current to transport the at least one analgesic or anesthetic active agent from the active agent reservoir, to the biological interface of the subject, and to administer a therapeutically effective amount of the at least one analgesic or anesthetic active agent to produce analgesic or anesthetic therapy in the subject for a limited period of time.

37. The method of claim 36 wherein the at least one analgesic or anesthetic active agent is selected from alfentanil, codeine, COX-2 inhibitors, opiates, opioid agonist, opioid antagonist, diamorphine, fentanyl, meperidine, methadone, morphine morphinomimetics, naloxone, nonsteroidal anti-inflammatory drugs (NSAIDs), oxycodone, remifentanil, sufentanil, and tricyclic antidepressants, or combinations thereof.

38. The method of claim 36, wherein the at least one analgesic or anesthetic active agent, further comprises:
one or more active agents selected from immuno-adjuvants, immuno-modulators, immuno-response agents, immuno-stimulators, specific immuno-stimulators, non-specific immuno-stimulators, and immuno-suppressants vaccines, agonist, antagonist, opioid agonist, opioid antagonist, antigens, adjuvants, immunological adjuvants, immunogens, tolerogens, allergens, toll-like receptor agonists, and toll-like receptor antagonists, or combinations thereof.

39. The method of claim 36 wherein applying a sufficient amount of current to transport the at least one analgesic or anesthetic active agent comprises:
providing sufficient voltage and current to deliver a therapeutically effective amount of the at least one analgesic or anesthetic active agent carried by the pharmaceutically acceptable vehicle comprising the at least one surfactant, the at least one nonpolar solvent, and the at least one polar agent; from the active agent reservoir to the biological interface of the subject.

40. The method of claim 36 wherein applying a sufficient amount of current to transport the at least one analgesic or anesthetic active agent comprises:
providing a sufficient voltage and current to the active electrode assembly to substantially achieve sustained-delivery or controlled-delivery of a therapeutically effective amount of the at least one analgesic or anesthetic active agent carried by the pharmaceutically acceptable vehicle comprising the at least one surfactant, the at least one nonpolar solvent, and the at least one polar agent; from the active agent reservoir to the biological interface of the subject.

41. An iontophoretic drug delivery device for providing transdermal delivery of one or more therapeutic active agents to a biological interface, comprising:
an active electrode assembly including at least one active electrode element; and
at least one active agent reservoir, the at least one active agent reservoir including a pharmaceutically acceptable vehicle comprising a plurality of first vesicles; wherein the plurality of first vesicles are selected from liposomes, nisomes, ethasomes, transfersomes, virosomes, cyclic oligosaccharides, non ionic surfactant vesicles, and phospholipid surfactant vesicles; at least some of the first vesicles including one or more therapeutic active agents; and the at least one active electrode element operable to provide an electromotive force to drive at least some of the pharmaceutically acceptable vehicle comprising the plurality of first vesicles; from the at least one active agent reservoir to the biological interface.

42. The iontophoretic drug delivery device of claim 41 wherein the one or more therapeutic active agents are selected from immuno-adjuvants, immuno-modulators, immuno-response agents, immuno-stimulators, specific immuno-stimulators, non-specific immuno-stimulators, and immuno-suppressants, or combinations thereof.

43. The iontophoretic drug delivery device of claim 41 wherein the one or more therapeutic active agents are selected from vaccines, agonist, antagonist, opioid agonist, opioid antagonist, antigens, adjuvants, immunological adjuvants, immunogens, tolerogens, allergens, toll-like receptor agonists, and toll-like receptor antagonists, or combinations thereof.

44. The iontophoresis device of claim 41 wherein the one or more therapeutic active agent are selected from centbucridine, tetracaine, Novocaine® (procaine), ambucaine, amolanone, amylcaine, benoxinate, betoxycaine, carticaine, chloroprocaine, cocaethylene, cyclomethycaine, butethamine, butoxycaine, carticaine, dibucaine, dimethisoquin, dimethocaine, diperodon, dyclonine, ecogonidine, ecognine, euprocin, fenalcomine, formocaine, hexylcaine, hydroxyteteracaine, leucinocaine, levoxadrol, metabutoxycaine, methyl chloride, myrtecaine, butamben, bupivicaine, mepivacaine, beta-adrenoceptor antagonists, opioid analgesics, butanilicaine, ethyl aminobenzoate, fomocine, hydroxyprocaine, isobutyl p-aminobenzoate, naepaine, octacaine, orthocaine, oxethazaine, parenthoxycaine, phenacine, phenol, piperocaine, polidocanol, pramoxine, prilocalne, propanocaine, proparacaine, propipocaine, pseudococaine, pyrrocaine, salicyl alcohol, parethyoxycaine, piridocaine, risocaine, tolycaine, trimecaine, tetracaine, anticonvulsants, antihistamines, articaine, cocaine, procaine, amethocaine, chloroprocaine, Lidocaine® (xylocaine), marcaine, chloroprocaine, etidocaine, prilocaine, lignocaine, benzocaine, zolamine, ropivacaine, and dibucaine, or combinations thereof.

45. The iontophoretic drug delivery device of claim 41 wherein the pharmaceutically acceptable vehicle comprising a plurality of first vesicles is formulated as a controlled-release formulation.

46. The iontophoresis device of claim 41 wherein a substantial portion of the plurality of first vesicles takes the form of liposomes.

47. The iontophoresis device of claim 41 wherein a substantial portion of the plurality of first vesicles includes one or more therapeutic active agents selected from amphiphilic active agents, lipophilic active agents, hydrophilic active agents, and charged hydrophilic active agents, or combinations thereof.

48. The iontophoresis device of claim 41 wherein a substantial portion of the plurality of first vesicles takes the form of unilamella or multilamellar vesicles

49. The iontophoresis device of claim 41 wherein a substantial portion of the plurality of first vesicles includes at least one vesicle bilayer and an encapsulated aqueous compartment.

50. The iontophoresis device of claim 49 wherein a substantial portion of the plurality of first vesicles includes at least a first therapeutic active agent in the encapsulated aqueous compartment, and a second therapeutic active agent associated with the at least on vesicle bilayer; the first active agent selected from one or more hydrophilic active agents and charged hydrophilic active agents, the second therapeutic active agent selected from amphiphilic active agents, and lipophilic active agents.

51. The iontophoresis device of claim 41 wherein at least 10% of the plurality of first vesicles includes a first active agent.

52. The iontophoresis device of claim 41 wherein at least 30% of the plurality of first vesicles includes a first active agent.

53. The iontophoresis device of claim 41 wherein at least 60% of the plurality of first vesicles includes a first active agent.
